(19) United States
(12) Patent Application Publication
(10) Pub. No.: US 2012/0115895 A1
(43) Pub. Date: May 10, 2012

(54) TRICYCLIC INDOLE-DERIVED SPIRO DERIVATIVES AS CRTH2 MODULATORS

(75) Inventors: Stefano Crosignani, St.Genis-Pouilly (FR); Catherine Jorand-Lebrun, Contamine-Sarzin (FR); Tania Grippi-Vallotton, Puplinge (CH)

(73) Assignee: Merck Serono S.A., Coinsins, Vaud (CH)

(21) Appl. No.: 13/383,857

(22) PCT Filed: Jul. 14, 2010
(86) PCT No.: PCT/EP2010/060154

§ 371 Date: Jan. 13, 2012

Related U.S. Application Data
(60) Provisional application No. 61/229,373, filed on Jul. 29, 2009.

(30) Foreign Application Priority Data
Jul. 15, 2009 (EP) 09165567.0

(51) Int. Cl. A61K 31/4439; A61K 31/422; A61K 31/4188; C07D 487/10; A61P 19/02; A61P 29/00; A61P 37/08; A61P 11/06; A61P 1/00; A61K 31/427; A61P 25/00

(52) U.S. Cl. 514/278; 514/365; 514/378; 514/389; 546/15; 548/147; 548/249; 548/301.1

(57) ABSTRACT

The present invention relates to compounds of formula (I) for use as pharmaceutical active compounds, as well as pharmaceutical formulations containing the same, for the treatment of allergic diseases. The compounds according to Formula (I) are suitable as modulators of CRTH2. The invention provides Spiro derivatives according to Formula (I) and related formulae that are useful in the treatment and/or prevention of diseases selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drag-induced exanthems (e.g., toxic epidermal necrolysis or Lyell's syndrome/Stevens-Johnson syndrome/drug hypersensitivity syndrome), photodermatosis or polymorphous light emption (e.g., photoirritant contact dermatitis, photoallergy contact dermatitis, chronic actinic dermatitis), and myositis neurodegenerative disorders such as neuropatic pain and other diseases with an inflammatory component such as rheumatoid arthritis, multiple sclerosis, osteoarthritis, and inflammatory bowel disease (IBD).

The present invention relates to compounds of formula (I) for use as pharmaceutical active compounds, as well as pharmaceutical formulations containing such spiro derivatives. Specifically, the invention relates to spiro derivatives of Formula (I):

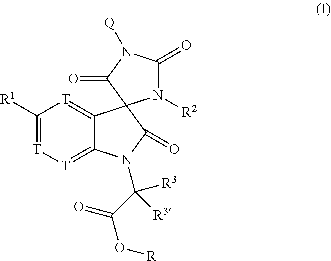

Wherein

### R¹ is H, Hal, A, CN, OA, CF₃, OCF₃,

### R² is A

R³, R³′ are independently from one another H or A

### R⁴ is H or A

Q is A, —(CH₂)ₙ—Ar, —(CH₂)ₙHet, —(CH₂)ₚ—(CHR¹¹)_{q}—(CH₂)ᵣ—Ar, or —(CH₂)ₚ—(CHR¹¹)_{q}—(CH₂)ᵣ-Het
p and r are independently from one another 0, 1, 2 3 or 4,
q is 1 or 2,
R¹¹ denotes H, A, CN, OR⁶, Hal, Ar, or Het,
n is 1, 2, 3, or 4

### T is CR⁵ or N

### R⁵ is H, Hal, A, CN, OA, CF₃, OCF₃.

A is branched or linear alkyl having 1 to 12 C-atoms, wherein one or more, preferably 1 to 7H-atoms may be replaced by Hal, OR⁶, CN, N(R⁶)₂, cycloalkyl, Ar or Het, and wherein one or more, preferably 1 to 7 CH₂-groups may be replaced by O, NR⁶, CON(R⁶)₂ or S and/or by CH═CH— or —C≡C— groups, or denotes cycloalkylen or cycloalkylalkylen having 3 to 7 ring C atoms.

### Hal is F, Cl, Br or I,

Ar denotes a monocyclic or bicyclic, unsaturated or aromatic carbocyclic ring having 6 to 14 carbon atoms which may be unsubstituted or monosubstituted, disubstituted or trisubstituted by Hal, A, CH₂OA, —CH₂OR⁶, OR⁶, CF₃, OCF₃, N(R⁶)₂, NO₂, CN, NR⁶COA, NR⁶SO₂A, COR⁶, SO₂N(R⁶)₂, SOA, SO₂A, Het, or Ar′.
Ar′ denotes a monocyclic or bicyclic, unsaturated or aromatic carbocyclic ring having 6 to 14 carbon atoms which may be unsubstituted or monosubstituted, disubstituted or trisubstituted by Hal, A, —CH₂OA, —CH₂OR⁶, —OR⁶, —CF₃, —OCF₃,
Het denotes a monocyclic or bicyclic, saturated, unsaturated or aromatic heterocyclic ring, having 1 to 4 N, O and/or S atoms, which may be unsubstituted or monosubstituted, disubstituted or trisubstituted by Hal, A, CH₂OA, OR⁶, CF₃, OCF₃, N(R⁶)₂, NO₂, CN, NR⁶COA, NR⁶SO₂A, COR⁶, SO₂N(R⁶)₂, SOA, SO₂A, Ar.

### R⁶ is H or A

As well as pharmaceutically usable derivatives, enantiomers, diastereoisomers, tautomers, salts, solvates and mixtures thereof in all ratios.

Said derivatives are useful for the treatment and/or prevention of allergic diseases and inflammatory dermatoses. Specifically, the present invention is related to the use of spiro derivatives for the modulation of CRTH2 activity.

The present invention furthermore relates to methods of the preparation of spiro derivatives.

The present invention also relates to a kit or a set consisting of separate packs of
(b) (a) an effective amount of a compound according to formula (I) and/or pharmaceutically usable derivatives, tautomers, salts, solvates and stereoisomers thereof, including mixtures thereof in all ratios,
   and
(c) an effective amount of a further medicament active ingredient.

### BACKGROUND OF THE INVENTION

Prostaglandin D2 (PGD2) has long been associated with inflammatory and atopic conditions, specifically allergic diseases such as asthma, rhinitis and atopic dermatitis (Lewis et al. (1982) J. Immunol. 129, 1627). PGD2 belongs to a class of compounds derived from the 20-carbon fatty acid skeleton of arachidonic acid. In response to an antigen challenge, PGD2 is released in large amounts into the airway as well as to the skin during an acute allergic response. The DP receptor, which is a member of the G-protein coupled receptor (GPCR) subfamily, has long been thought to be the only receptor of PGD2. DP's role in allergic asthma has been demonstrated with DP deficient mice (Matsuoka et al. (2000) Science 287, 2013-2017). However, despite intense interest in the role of PGD2 in the inflammatory response, a direct link between DP receptor activation and PGD2-stimulated eosinophil migration has not been established (Woodward et al. (1990) Invest. Ophthalomol. Vis. Sci. 31, 138-146; Woodward et al. (1993) Eur. J. Pharmacol. 230, 327-333). More recently, another G-protein coupled receptor, referred to as “Chemoattractant Receptor-Homologous molecule expressed on T-Helper 2 cells” (CRTH2) (Nagata et al. (1999) J. Immunol. 162, 1278-1286, Hirai et al. (2001) J. Exp. Med. 193, 255-261) has recently been identified as a receptor for PGD2 and this discovery has begun to shed light on the mechanism of action of PGD2. CRTH2, which is also referred to as DP2, GPR44 or DLIR, shows little structural similarity with the DP receptor and other prostanoid receptors. However, CRTH2 possesses similar affinity for PGD2. Among peripheral blood T lymphocytes, human CRTH2 is selectively expressed on Th2 cells and is highly expressed on cell types associated with allergic inflammation such as eosinophils, basophiles and Th2 cells. In addition, CRTH2 mediates PGD2 dependent cell migration of blood eosinophils and basophiles. Furthermore, increased numbers of circulating T cells expressing CRTH2 have been correlated with the severity of atopic dermatitis (Cosmi et al. (2000) Eur. J. Immunol. 30, 2972-2979). The interaction of CRTH2 with PGD2 plays a critical role in the allergen-induced recruitment of Th2 cells in the target tissues of allergic inflammation. Compounds that inhibit the binding of CRTH2 and PGD2 should therefore be useful for the treatment of allergic diseases.

Allergic disease, like asthma, and inflammatory dermatoses represent a major class of complex, and typically chronic, inflammatory diseases that currently affect about 10% of the population and that number appears to be increasing (Bush, R. K., Georgitis J. W., Handbook of asthma and rhinitis 1st ed. (1997), Abingdon: Blackwell Science. 270). Atopic dermatitis is a chronic skin disease, wherein the skin becomes extremely itchy. It accounts for 10 to 20 percent of all visits to dermatologists. The increasing incidence of allergic diseases and inflammatory dermatoses worldwide underscores the need for new therapies to effectively treat or prevent these diseases. Currently, numerous classes of pharmaceutical agents are widely used to treat these diseases, for example, antihistamines, decongestants, anticholinergics, methylxanthines, cromolyns, corticosteroids, and leukotriene modulators. However, the usefulness of these agents is often limited by side effects and low efficacy.

WO 2006125784 provides similar spiroderivatives as CRTH2 modulators.

One aim of the present invention is to provide compounds having improved biological and/or physico-chemical properties. In particular, compounds of the present invention have improved bioavailability.

The invention further provides a pharmaceutical composition comprising a compound of Formula (I), together with a pharmaceutically acceptable excipient or carrier.

The invention further relates to the use of compounds of Formula I for the preparation of a medicament for the treatment and/or prevention of diseases selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis, neurodegenerative disorders such as neuropatic pain, and other diseases with an inflammatory component such as rheumatoid arthritis, multiple sclerosis, osteoarthritis, and inflammatory bowel disease (IBD) and other diseases and disorders associated with CTRH2 activity. Specifically the present invention is related to the use of compounds of Formula (I) for the modulation of CRTH2 activity.

The invention further relates to a method for treating and/or preventing a patient suffering from a disease selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis, neurodegenerative disorders such as neuropatic pain and other diseases with an inflammatory component such as rheumatoid arthritis, multiple sclerosis, osteoarthritis, and inflammatory bowel disease (IBD) and other diseases and disorders associated with CTRH2 activity, by administering a compound according to Formula (I).

The invention further relates to the use of compounds of Formula (I) for the preparation of a pharmaceutical composition.

The invention finally relates to novel compounds of Formula (I) as well as to methods to synthesize compounds of Formula (I).

In a preferred embodiment, the present invention provides compounds of formula (I) wherein R⁴ is H.

In another preferred embodiment, the present invention provides compounds of formula (I) wherein R¹ is halogen, methyl, CN, CF₃, OCF₃, R² is an alkyl having 1 to 6 carbon atoms or a group —CH₂—R⁷ wherein R⁷ is —CH₂F, —CH₂OCH₃, —CH₂CONH₂, pyridine, or CN. R³, R³′ and R⁴ are H, T is CR⁵ whereby R⁵ is H.

Most preferably, the present invention provides compounds of formula (I) wherein

R¹ is halogen, R² is an alkyl having 1 to 6 carbon atoms, R⁴ is H and T is CR⁵ whereby R⁵ is H.

In a preferred embodiment, the present invention provides compounds of Formula (Ia)

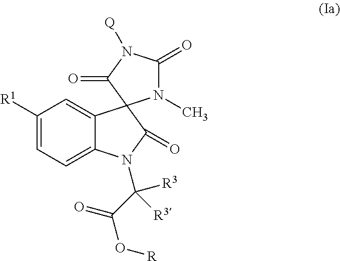

Wherein R¹, Q, R³, R³′ and R⁴ are as defined above.

The compounds of Formula (Ia) exist as enantiomers (Aa) and (Ba) as shown below:

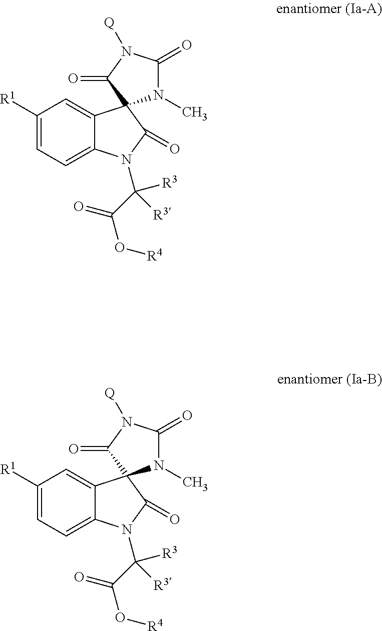

In another preferred embodiment, the present invention provides compounds of Formula (Ib)

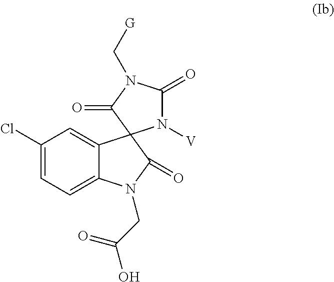

Wherein G is Ar or Het

And wherein V is an alkyl having 1 to 6 carbon atoms, preferably V is methyl.

Compounds of Formula (Ib) also exists as enantiomers (Ab) and (Bb)

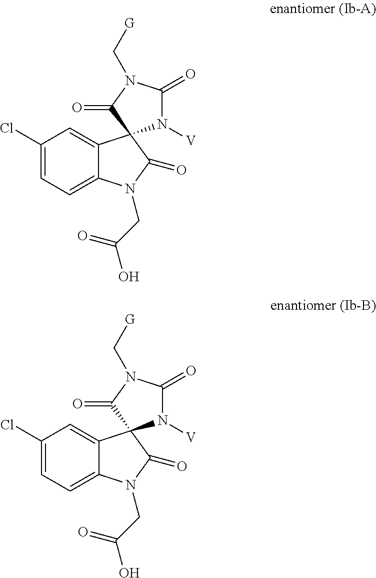

Pure enantiomers as well as racemic mixtures of compounds of Formulae (I), (Ia), (Ib) and (Ic) are within the scope of the present invention.

In one embodiment, compounds of the present invention are enantiomerically enriched. In particular, they exhibit an optical rotatory power either positive or negative.

In a preferred embodiment, the optical rotatory power is negative.

In another preferred embodiment, the optical rotatory power is positive.

Compounds of Formula (I′), wherein R¹, R², R³, R³′, Q, and T are defined as above, and wherein R⁴ is A, can be obtained from a compound of Formula (III) as outlined in Scheme 1.

The first step consists in the reaction of a compound of Formula (III), wherein R¹, R³, R³′, and T are defined as above, with a compound R²—W, wherein R² is defined as above and W is a suitable leaving group, such as but not limited to Cl, Br, I, OMs, OTf and others known to those skilled in the art. The reaction is performed in the presence of a suitable base, such as but not limited to K₂CO₃, Na₂CO₃, NaHCO₃, NaOH, KOH, KOtBu, NaH, LDA, LiHMDS, BuLi, preferably K₂CO₃, Na₂CO₃, NaHCO₃, in the presence or absence of NaI or KI (in catalytic or stoichiometric amount) in a suitable solvent such as but not limited to THF, dioxane, DMF, DMSO, preferably DMF, at a temperature between −80° C. to 160° C., preferably at about 25° C., for a few hours, e.g. one hour to 48 h.

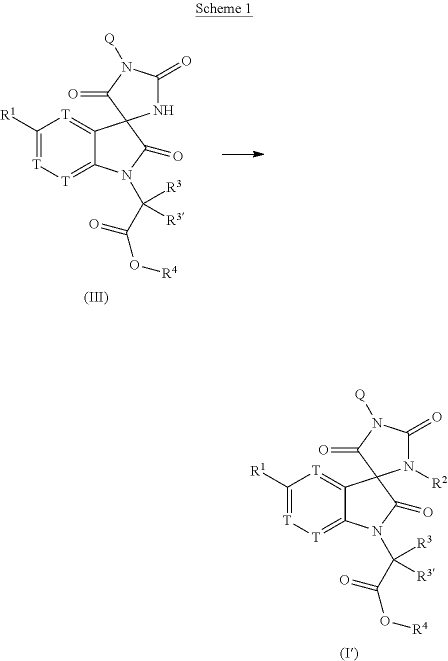

Compounds of Formula (I″), wherein R¹, R², R³, R³′, Q, and T are defined as above, and wherein R⁴ is H, can be obtained from a compound of Formula (I*) wherein R⁴ is A, preferably a C₁-C₆alkyl, as outlined in Scheme 2

This reaction can be achieved using conditions and methods well known to those skilled in the art for the conversion of an ester to a carboxylic acid, such as but not limited to treatment with a base, such as KOH, LiOH, NaOH, K₂CO₃ or an appropriate acid, such as trifluoroacetic acid or hydrochloric acid, in the presence or absence of water, in the presence of a suitable solvent such as DCM, dioxane, THF at a temperature between about 20° C. to about 100° C., preferably at about 20° C., for a few hours, e.g. one hour to 24 h.

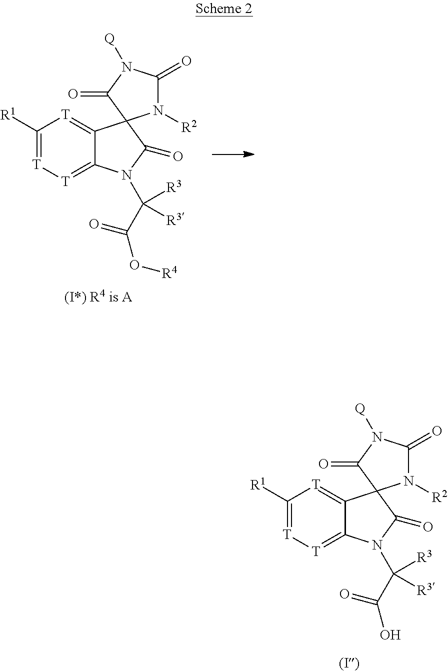

Alternatively, compounds of Formula (I′), wherein R¹, R², R³, R³′, and T are defined as above and wherein R⁴ is A, can be prepared as outlined in Scheme 3. Compounds of Formula (IV), wherein R¹, R³, R³′, and T are defined as above and R⁴ is A, can be selectively reacted with a compound R²—W, wherein R² is defined as above and W is a suitable leaving group, such as but not limited to Cl, Br, I, OMs, OTf and others known to those skilled in the art. The desired product of Formula (V) can be obtained using an appropriate amount (usually two equivalents) of an appropriate base, such as but not limited to LDA, LiHMDS, BuLi, in a suitable solvent such as but not limited to THF or dioxane, between −80° C. to 20° C. for a few hours, e.g. one hour to 24 h.

The second step consists in the reaction of a compound of Formula (V), wherein R¹, R², R³, R³′ and T are defined as above, and wherein R⁴ is A, with a compound Q-W, wherein Q is defined as above and W is a suitable leaving group, such as but not limited to Cl, Br, I, OMs, OTf and others known to those skilled in the art. The reaction is performed in the presence of a suitable base, such as but not limited to K₂CO₃, Na₂CO₃, NaHCO₃, NaOH, KOH, KOtBu, NaH, LDA, LiHMDS, BuLi, preferably K₂CO₃, Na₂CO₃, NaHCO₃ in the presence or absence of NaI or KI (in catalytic or stoichiometric amount), in a suitable solvent such as but not limited to THF, dioxane, DMF, DMSO, preferably DMF, at a temperature between −80° C. to 160° C., preferably 25° C. for a few hours, e.g. one hour to 48 h.

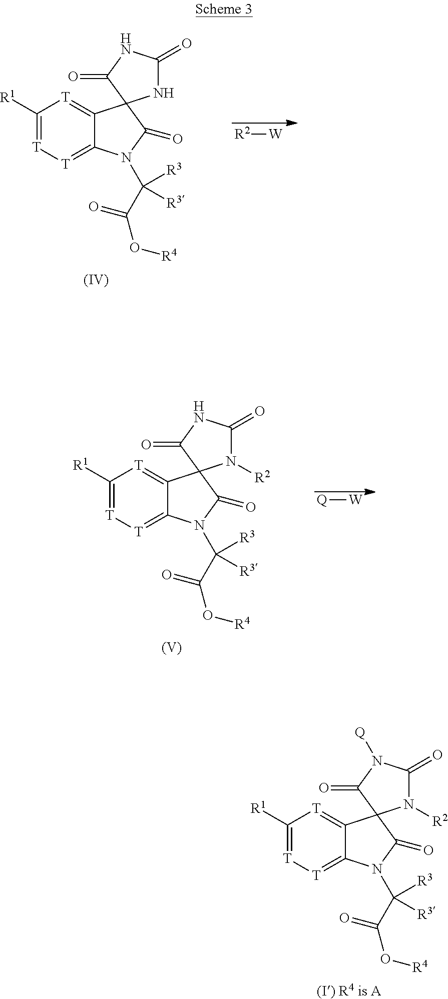

In a specific embodiment the present invention provides a process for preparing compounds of formula (I) and related formulae, comprising the step of regioselectively reacting compound of formula (IV) with R²—W, to afford compound of formula (V),

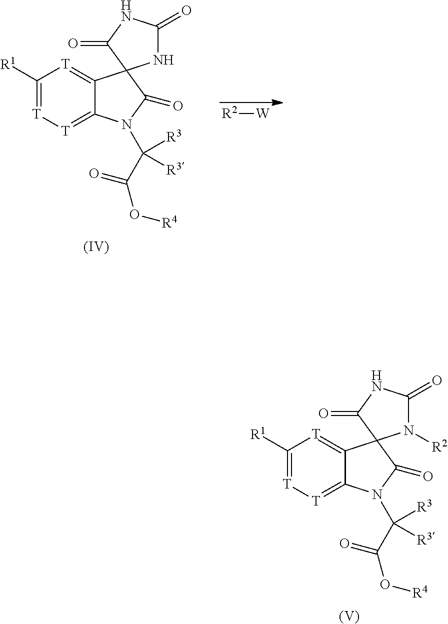

Wherein R¹, T, R², R³, R³′ and R⁴ are as above defined and

Wherein W is a leaving group selected from Cl, Br, I, OMs, OTf.

Having a chiral centre, the compounds of Formula (I) exists as either racemic or in enantiomerically enriched form. Racemic compounds of Formula (I) can be separated into the two enantiomers by methods well known to those skilled in the art, such as but not limited to using a chromatographic separation on a chiral stationary phase, or using a chiral mobile phase, as well as forming diastereomeric salts, adducts, esters or such, as it is well described in the literature. In addition, the separation of the enantiomers can be accomplished on the intermediates of Formulas III-V, using such methods as described above, which will afford, after the steps described above, products of Formula (I) in an enantiomerically enriched form.

In one embodiment, the enantiomerically enriched compound of the present invention is the first eluted from the racemic mixture separated by a chiral chromatography.

In another embodiment, the enantiomerically enriched compound of the present invention is the second eluted from the racemic mixture separated by a chiral chromatography.

“cycloalkyl” denotes a monovalent saturated carbocyclic ring having 3 to 7 carbon atoms. Preferred cycloalkyl groups are cyclopropyl, cyclobutyl, cyclopentyl and cyclohexyl.

“cycloalkylen” denotes a divalent saturated carbocyclic ring having 3 to 7 carbon atoms.

“cycloalkylalkylen” denotes a carbon chain having 1 to 6 carbon atoms wherein 1H atom is substituted by a cycloalkyl group.

In the compounds of Formula (I), wherein a substituent occurs more than once, such as T, each of them has the meaning hereby defined, independently from one another.

The group A preferably denotes a branched or linear alkyl having 1 to 6 C-atoms, wherein one or more, preferably 1 to 7H-atoms may be replaced by Hal, OR⁶, CN, N(R⁶)₂, Ar or Het, and wherein one or more, preferably 1 to 3 CH₂-groups may be replaced by O, NR⁶, or CON(R⁶)₂.

Q is preferably a branched or linear alkyl having 1 to 6 carbon atoms, wherein 1 to 2H-atoms may be replaced by Ar or Het and more preferably Q is a group —(CH₂)ₙ—Ar, —(CH₂)ₙHet, wherein n is as defined above.

In another preferred embodiment, Q also denotes —(CH₂)ₚ—(CHR¹¹)_{q}—(CH₂)ᵣ—Ar, or —(CH₂)ₚ—(CHR¹¹)_{q} (CH₂)ᵣ-Het wherein p and r are independently from one another 0, 1, 2, 3 or 4, and wherein r is 1 or 2, and wherein R¹¹ denotes H, an alkyl having 1 to 6 carbon atoms, CN, OR⁶, Hal, whereby R⁶ is as above defined.

Most preferably, Q is selected from the following groups:

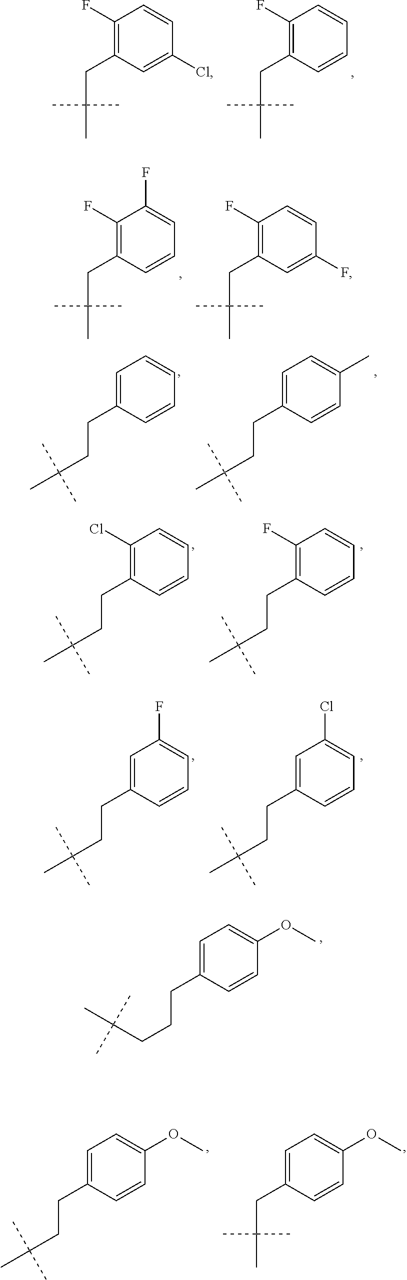

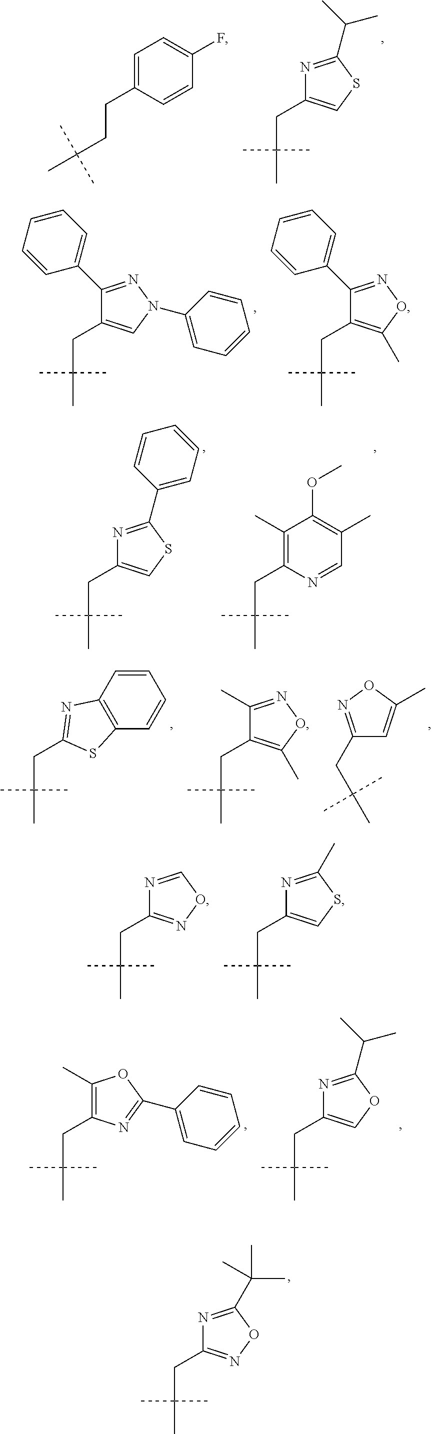

Ar preferably denotes a monocyclic or bicyclic, unsaturated or aromatic carbocyclic ring having 6 to 14 carbon atoms which may be unsubstituted or monosubstituted, disubstituted by Hal, A, —CH₂OR⁶, OR⁶, CF₃, OCF₃, CN, Het, or Ar′.

More preferably Ar denotes the following group:

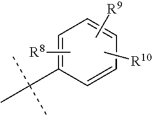

Wherein R⁸, R⁹ and R¹⁰ are independently selected from H, A, Hal, Het, linear or branched alkyl having 1 to 6 carbon atoms, Ar′, OR⁶, CN, CF₃, and OCF₃.

Most preferably, Ar denotes one of the following groups.

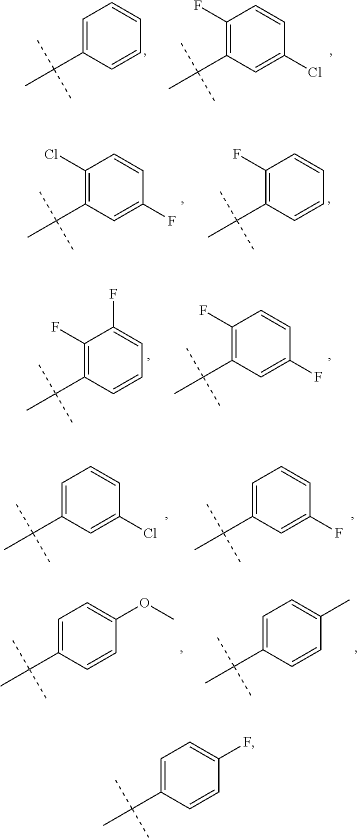

Ar′ preferably denotes a phenyl group unsubstituted or substituted with 1 or 2 groups selected from Hal, A and an alkyl having 1 to 6 carbon atoms.

Het preferably denotes a monocyclic or bicyclic, saturated, unsaturated or aromatic heterocyclic ring, having 1 to 2 N atoms and/or 1 O or S atom, which may be unsubstituted or monosubstituted, disubstituted or trisubstituted by Hal, A, CH₂OA, OR⁶, CF₃, OCF₃, CN, or Ar.

More preferably, Het denotes, not withstanding further substitutions, for example, 2- or 3-furyl, 2- or 3-thienyl, 1-, 2- or 3-pyrrolyl, 1-, 2-, 4- or 5-imidazolyl, 1-, 3-, 4- or 5-pyrazolyl, 2-, 4- or 5-oxazolyl, 3-, 4- or 5-isoxazolyl, 2-, 4- or 5-thiazolyl, 3-, 4- or 5-isothiazolyl, 2-, 3- or 4-pyridyl, 2-, 4-, 5- or 6-pyrimidinyl, furthermore preferably 1,2,3-triazol-1-, -4- or -5-yl, 1,2,4-triazol-1-, -3- or -5-yl, 1- or 5-tetrazolyl, 1,2,3-oxadiazol-4- or -5-yl, 1,2,4-oxadiazol-3- or -5-yl, 1,3,4-thiadiazol-2- or -5-yl, 1,2,4-thiadiazol-3- or -5-yl, 1,2,3-thiadiazol-4- or -5-yl, 3- or 4-pyridazinyl, pyrazinyl, 1-, 2-, 3-, 4-, 5-, 6- or 7-indolyl, indazolyl, 4- or 5-isoindolyl, 1-, 2-, 4- or 5-benzimidazolyl, 1-, 3-, 4-, 5-, 6- or 7-benzopyrazolyl, 2-, 4-, 5-, 6- or 7-benzoxazolyl, 3-, 4-, 5-, 6- or 7-benzisoxazolyl, 2-, 4-, 5-, 6- or 7-benzothiazolyl, 2-, 4-, 5-, 6- or 7-benzisothiazolyl, 4-, 5-, 6- or 7-benz-2,1,3-oxadiazolyl, 2-, 3-, 4-, 5-, 6-, 7- or 8-quinolyl, 1-, 3-, 4-, 5-, 6-, 7- or 8-isoquinolyl, 3-, 4-, 5-, 6-, 7- or 8-cinnolinyl, 2-, 4-, 5-, 6-, 7- or 8-quinazolinyl, 5- or 6-quinoxalinyl, 2-, 3-, 5-, 6-, 7- or 8-2H-benzo-1,4-oxazinyl, furthermore preferably 1,3-benzodioxol-5-yl, 1,4-benzodioxane-6-yl, 2,1,3-benzothiadiazol-4- or -5-yl or 2,1,3-benzoxadiazol-5-yl.

The heterocyclic radicals may also be partially or fully hydrogenated. Het can thus also denote, for example, 2,3-dihydro-2-, -3-, -4- or -5-furyl, 2,5-dihydro-2-, -3-, -4- or -5-furyl, tetrahydro-2- or -3-furyl, 1,3-dioxolan-4-yl, tetrahydro-2- or -3-thienyl, 2,3-dihydro-1-, -2-, -3-, -4- or -5-pyrrolyl, 2,5-dihydro-1-, -2-, -3-, -4- or -5-pyrrolyl, 1-, 2- or 3-pyrrolidinyl, tetrahydro-1-, -2- or -4-imidazolyl, 2,3-dihydro-1-, -2-, -3-, -4- or -5-pyrazolyl, tetrahydro-1-, -3- or -4-pyrazolyl, 1,4-dihydro-1-, -2-, -3- or -4-pyridyl, 1,2,3,4-tetrahydro-1-, -2-, -3-, -4-, -5- or -6-pyridyl, 1-, 2-, 3- or 4-piperidinyl, 2-, 3- or 4-morpholinyl, tetrahydro-2-, -3- or -4-pyranyl, 1,4-dioxaneyl, 1,3-dioxane-2-, -4- or -5-yl, hexahydro-1-, -3- or -4-pyridazinyl, hexahydro-1-, -2-, -4- or -5-pyrimidinyl, 1-, 2- or 3-piperazinyl, 1,2,3,4-tetrahydro-1-, -2-, -3-, -4-, -5-, -6-, -7- or -8-quinolyl, 1,2,3,4-tetrahydro-1-, -2-, -3-, -4-, -5-, -6-, -7- or -8-isoquinolyl, 2-, 3-, 5-, 6-, 7- or 8-3,4-dihydro-2H-benzo-1,4-oxazinyl, furthermore preferably 2,3-methylenedioxyphenyl, 3,4-methylenedioxyphenyl, 2,3-ethylenedioxyphenyl, 3,4-ethylenedioxyphenyl, 3,4-(difluoromethylenedioxy)phenyl, 2,3-dihydrobenzofuran-5- or -6-yl, 2,3-(2-oxomethylenedioxy)phenyl or also 3,4-dihydro-2H-1,5-benzodioxepin-6- or -7-yl, furthermore preferably 2,3-dihydrobenzofuranyl or 2,3-dihydro-2-oxofuranyl.

The more preferred Het groups are selected from the following groups:

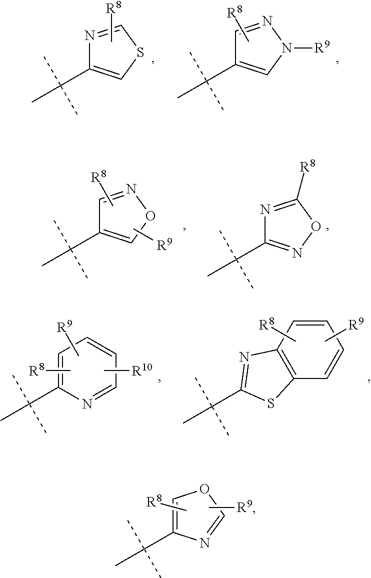

Wherein R⁸, R⁹ and R¹⁰ are independently selected from H, A, Hal, linear or branched alkyl having 1 to 6 carbon atoms, aryl, OR⁶, CN, CF₃, and OCF₃, whereby R⁶ is as defined above.

Most preferably, Het is selected from the following groups:

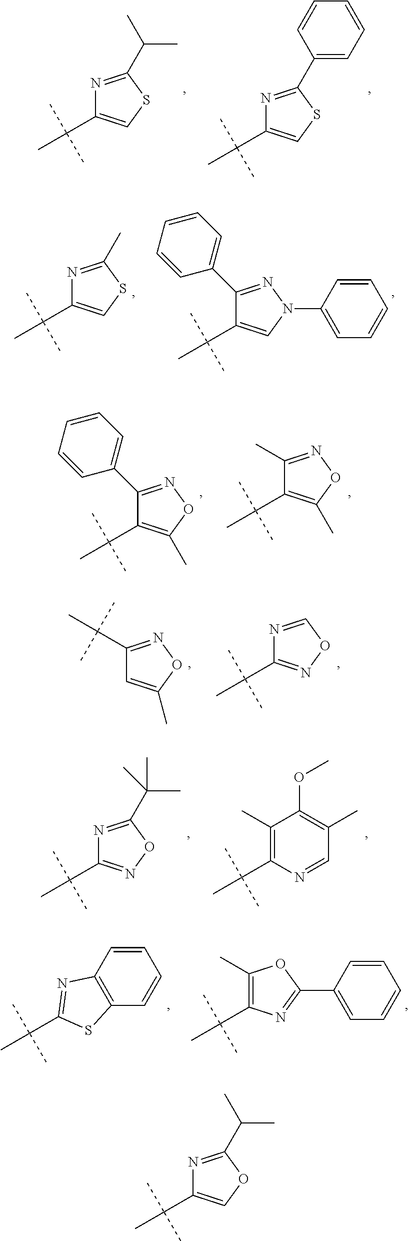

Preferred compounds of the present invention are represented by the following groups of formulae:

| Example | Formula |
|---|---|
| 1 | 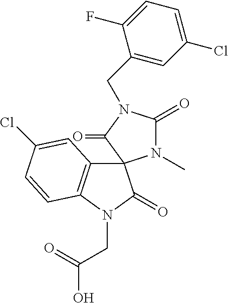 |
| 2 | 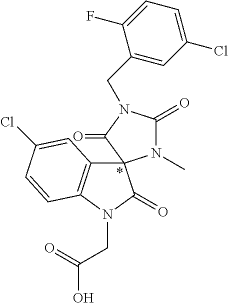 |
| 3 | 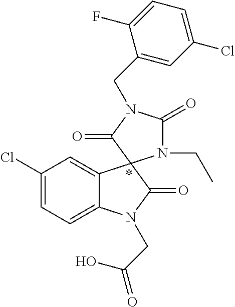 |
| 4 | 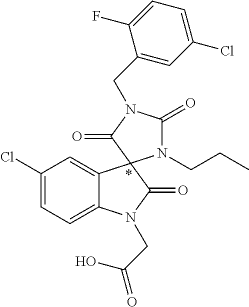 |
| 5 | 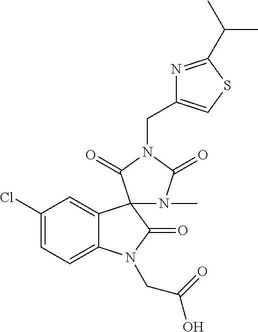 |
| 6 | 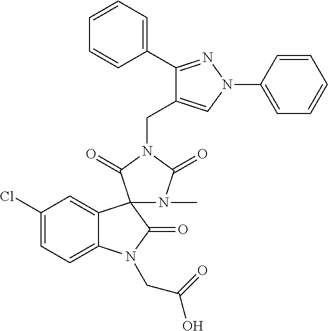 |
| 7 | 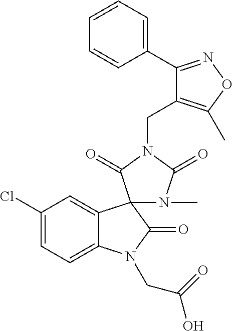 |
| 8 | 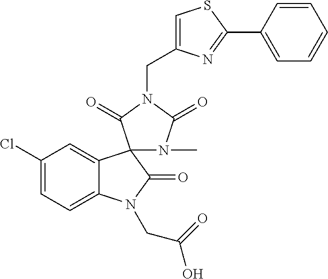 |
| 9 | 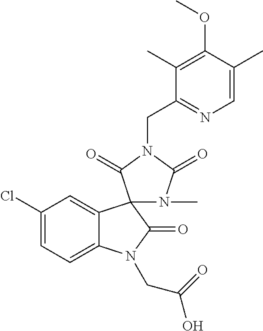 |
| 10 | 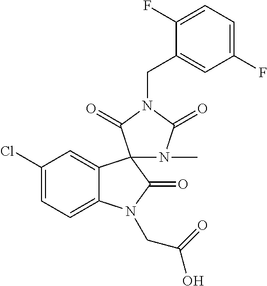 |
| 11 | 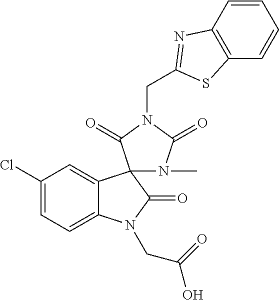 |
| 12 | 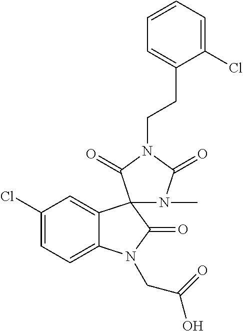 |
| 13 | 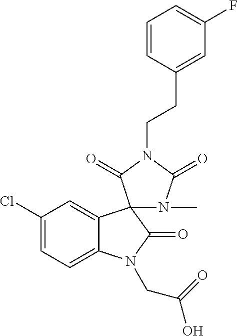 |
| 14 | 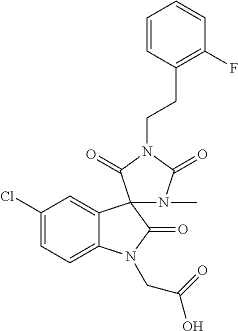 |
| 15 | 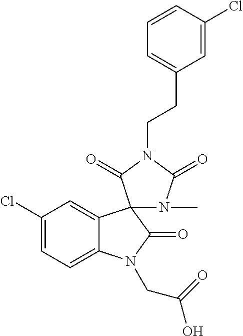 |

Wherein the symbol “*” indicates that the given formula represents one pure enantiomer or a enentiomerically enriched mixture of enantiomers.

Formula (I) and structures of Examples 1 to 15 include the mixtures of the following enantiomers in all ratios. Such mixtures may be racemic or enantiomerically enriched.

| Example | Formula |
|---|---|
| 2a | 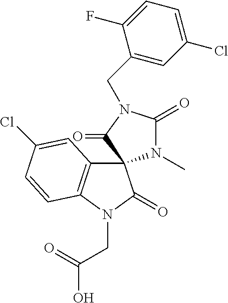 |
| 2b | 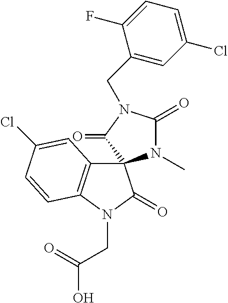 |
| 3a | 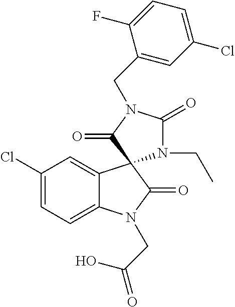 |
| 3b | 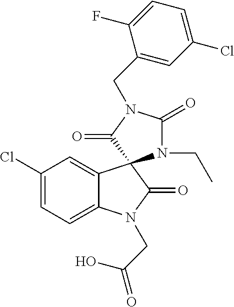 |
| 4a | 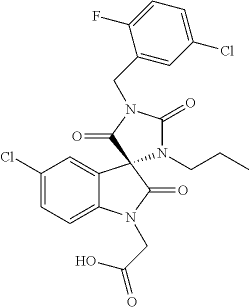 |
| 4b | 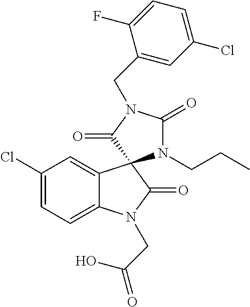 |
| 5a | 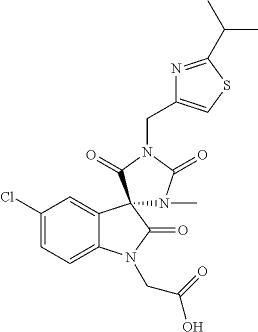 |
| 5b | 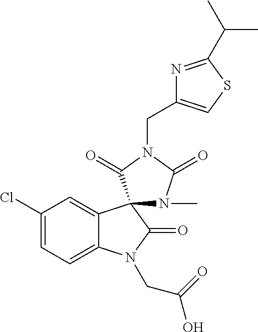 |
| 6a | 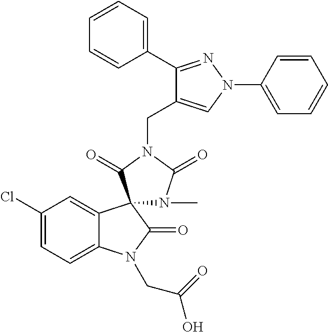 |
| 6b | 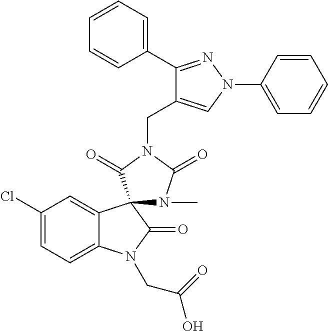 |
| 7a | 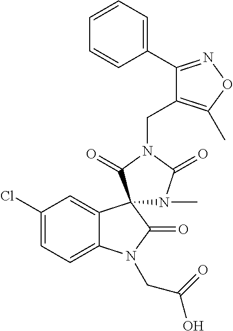 |
| 7b | 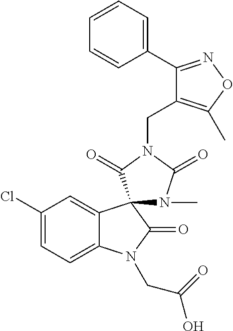 |
| 8a | 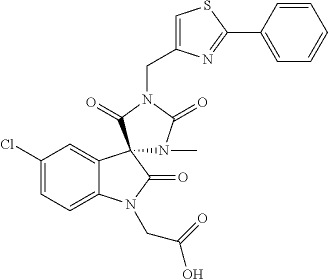 |
| 8b | 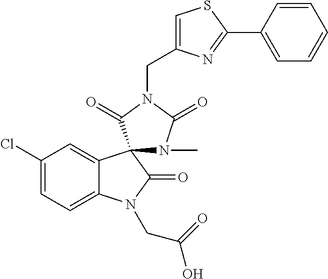 |
| 9a | 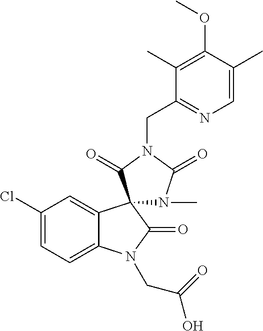 |
| 9b | 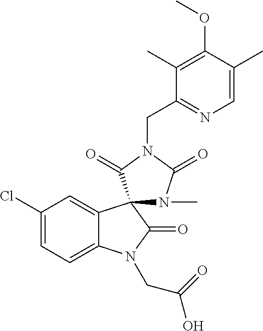 |
| 10a | 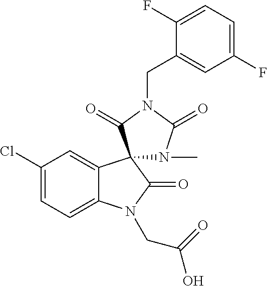 |
| 10b | 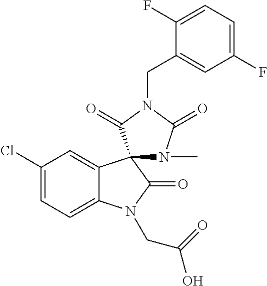 |
| 11a | 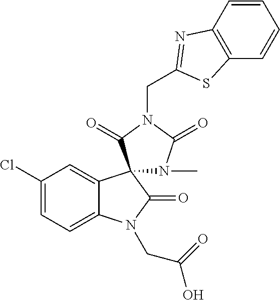 |
| 11b | 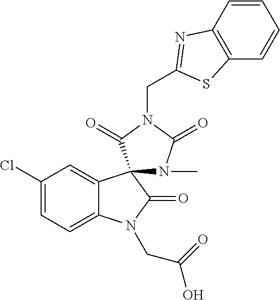 |
| 12a | 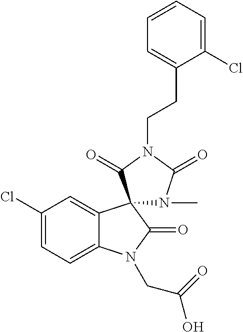 |
| 12b | 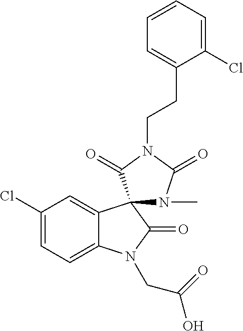 |
| 13a | 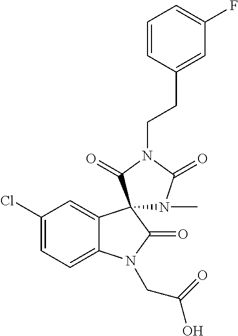 |
| 13b | 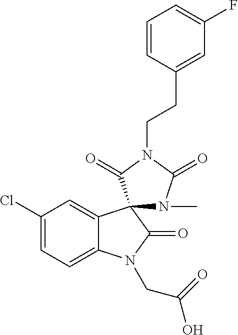 |
| 14a | 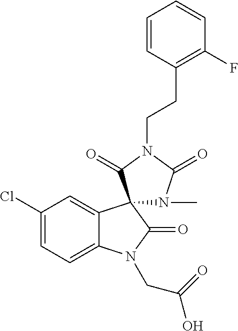 |
| 14b | 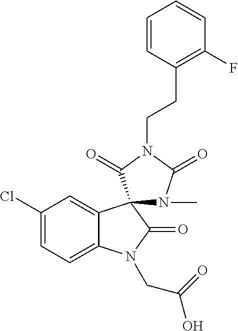 |
| 15a | 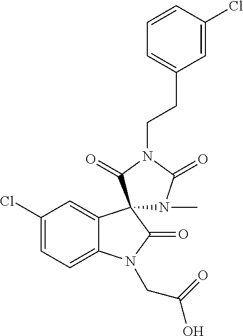 |
| 15b | 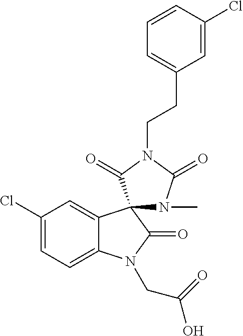 |

In another preferred embodiment, the present invention provides enantiomers I′a, I′b and I′c, characterized in that the optical rotatory power is positive.

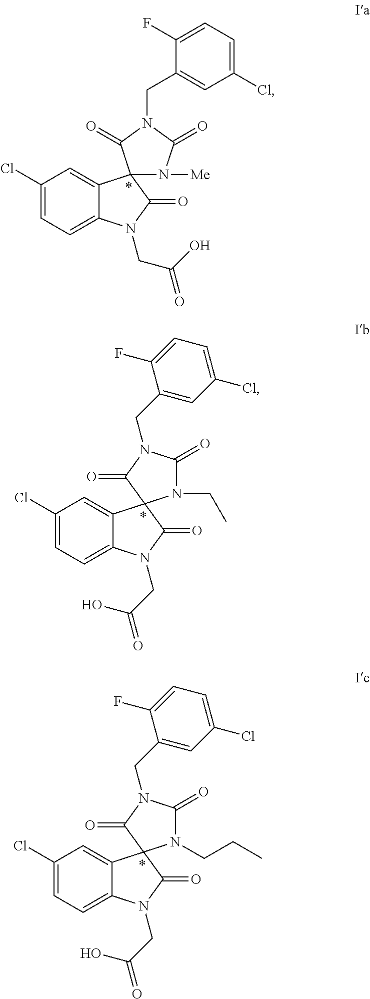

“enantiomerically enriched” is intended to define a compound of Formula (I) and related formulae wherein one enantiomer is present in excess compared to the other. The enantiomeric excess of an enantiomerically enriched compound is preferably between 20% and 100%, more preferably, the enentiomeric excess is more than 50%, and most preferably more than 95%, even more preferably above 98%.

“Pharmaceutically acceptable cationic salts or complexes” is intended to define such salts as the alkali metal salts, (e.g. sodium and potassium), alkaline earth metal salts (e.g. calcium or magnesium), aluminium salts, ammonium salts and salts with organic amines such as with methylamine, 2-N-morpholinoethanol, dimethylamine, trimethylamine, ethylamine, triethylamine, morpholine, N-Me-D-glucamine, N,N′-bis(phenylmethyl)-1,2-ethanediamine, ethanolamine, diethanolamine, ethylenediamine, N-methylmorpholine, piperidine, benzathine (N,N′-dibenzylethylenediamine), choline, ethylene-diamine, benethamine (N-benzylphenethylamine), diethylamine, piperazine, thromethamine (2-amino-2-hydroxymethyl-1,3-propanediol), procaine as well as amines of formula —NRR′R″ wherein R, R′, R″ is independently hydrogen, alkyl or benzyl.

“Pharmaceutically acceptable salts or complexes” refers to salts or complexes of the below-identified compounds of Formula I that retain the desired biological activity. Examples of such salts include, but are not restricted to, acid addition salts formed with inorganic acids (e.g. hydrochloric acid, hydrobromic acid, sulfuric acid, phosphoric acid, nitric acid, and the like), and salts formed with organic acids such as acetic acid, oxalic acid, tartaric acid, succinic acid, malic acid, fumaric acid, maleic acid, ascorbic acid, benzoic acid, tannic acid, pamoic acid, alginic acid, polyglutamic acid, naphthalene sulfonic acid, naphthalene disul-fonic acid, and poly-galacturonic acid. Said compounds can also be administered as pharmaceutically acceptable quaternary salts known by a person skilled in the art, which specifically include the quarternary ammonium salt of the Formula —NRR′R″+Z—, wherein R, R′, R″ is independently hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, fumarate, citrate, tartrate, ascorbate, cinnamoate, mandeloate, and diphenylacetate).

“Pharmaceutically active derivative” or “pharmaceutically usable derivative” refers to any compound that, upon administration to the recipient, is capable of providing directly or indirectly, the activity disclosed herein.

Throughout the specification, the term leaving group preferably denotes Cl, Br, I or a reactively modified OH group, such as, for example, an activated ester, an imidazolide or alkylsulfonyloxy having 1-6 carbon atoms (preferably methylsulfonyloxy or trifluoromethylsulfonyloxy) or arylsulfonyloxy having 6-10 carbon atoms (preferably phenyl- or p-tolylsulfonyloxy).

Radicals of this type for activation of the carboxyl group in typical acylation reactions are described in the literature (for example in the standard works, such as Houben-Weyl, Methoden der organischen Chemie [Methods of Organic Chemistry], Georg-Thieme-Verlag, Stuttgart).

Activated esters are advantageously formed in situ, for example through addition of HOBt or N-hydroxysuccinimide.

The term “solvates” is taken to mean adductions of inert solvent molecules onto the compounds which form owing to their mutual attractive force. Solvates are, for example, mono- or dihydrates or alcoholates.

The formula (I) and related formulae also encompass mixtures of the compounds of the formula (I), for example mixtures of two enantiomers or diastereomers, for example in the ratio 1:1, 1:2, 1:3, 1:4, 1:5, 1:10, 1:100 or 1:1000.

In a first aspect, the invention provides spiro derivatives according to Formula (I) and related formulae that are useful in the treatment and/or prevention of diseases selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis neurodegenerative disorders such as neuropatic pain and other diseases with an inflammatory component such as rheumatoid arthritis, multiple sclerosis, osteoarthritis, and inflammatory bowel disease (IBD).

In one embodiment the compounds according to Formula (I) are suitable as modulators of CRTH2. Therefore, the compounds of the present invention are also particularly useful for the treatment and/or prevention of disorders, which are mediated by CRTH2 activity. Said treatment involves the modulation of CRTH2 in mammals and particular in humans. The modulators of CRTH2 are selected from the group consisting of an inverse agonist, an antagonist, a partial agonist and an agonist of CRTH2.

In one embodiment, the modulators of CRTH2 are inverse agonists of CRTH2.

In another embodiment, the modulators of CRTH2 are antagonists of CRTH2.

In another embodiment, the modulators of CRTH2 are partial agonists of CRTH2.

In another embodiment, the modulators of CRTH2 are agonists of CRTH2.

The compounds according to Formula (I) are suitable for use as a medicament.

Compounds of Formula (I) include also their geometrical isomers, their optically active forms as enantiomers, diastereomers, its racemate forms, as well as pharmaceutically acceptable salts thereof,

In a second aspect, the invention provides the use of a spiro derivative according to Formula (I) and related formulae, for the preparation of a medicament for the treatment and/or prevention of a disease selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis; photoallergic contact dermatitis; chronic actinic dermatitis), and myositis, neurodegenerative disorders such as neuropatic pain and other diseases with an inflammatory component such as rheumatoid arthritis, multiple sclerosis, osteoarthritis, and inflammatory bowel disease (IBD) and other diseases and disorders associated with CTRH2 activity.

In a third aspect, the invention provides a method for treating and/or preventing a patient suffering from a disease selected from allergic diseases such as allergic asthma, allergic rhinitis, allergic conjunctivitis, and inflammatory dermatoses such as atopic dermatitis, contact hypersensitivity, allergic contact dermatitis, chronic urticaria/chronic idiopathic/autoimmune urticaria, drug-induced exanthems (e.g. toxic epidermal necrolysis or Lyell's syndrome/Stevens-Johnson syndrome/drug hypersensitivity syndrome), photodermatosis or polymorphous light eruption (e.g. photo-irritant contact dermatitis, photoallergic contact dermatitis, chronic actinic dermatitis), and myositis, neurodegenerative disorders such as neuropatic pain and other diseases with an inflammatory component such as rheumatoid arthritis, multiple sclerosis, osteoarthritis, and inflammatory bowel disease (IBD) and other diseases and disorders associated with CTRH2 activity, by administering a compound according to Formula (I) or related formulae.

The term “preventing”, as used herein, should be understood as partially or totally preventing, inhibiting, alleviating, or reversing one or more symptoms or cause(s) of allergic disease or inflammatory dermatitis.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

In a fourth aspect, the invention provides a pharmaceutical composition comprising a spiro derivative according to Formulae (I) or related formulae, together with a pharmaceutically acceptable excipient or carrier.

In a fifth aspect, the invention provides a pharmaceutical composition comprising a compound according to Formulae (I) or related formulae, together with a biologically active compound. In particular, the pharmaceutical composition contains a compounds of Formula (I) in combination with an anti-allergic drug.

In another embodiment, the pharmaceutical composition contains a compound of Formula (I) in combination with an antihistamine, a decongestant, an anticholinergic, a methylxanthine, a cromolyn, a corticosteroid or a leukotriene modulator.

In another embodiment, the pharmaceutical composition contains a compound of Formula (I) in combination with a drug used in the treatment of disease or disorder associated with CTRH2 activity.

In a sixth aspect, the present invention provides a method of reducing the dose of an anti-allergic drug. In particular, the present invention provides a mean of reducing the dose of antihistamines, decongestants, anticholinergics, methylxanthines, cromolyns, corticosteroids or leukotriene modulators.

In another embodiment, the present invention provides a mean to decrease the dose of drug used in the treatment of disease or disorder associated with CTRH2 activity.

The compounds of the invention are typically administered in form of a pharmaceutical composition. Such compositions can be prepared in a manner well known in the pharmaceutical art and comprise at least one active compound. Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of these inventions can be administered by a variety of routes including oral, rectal, transdermal, subcutaneous, intravenous, intramuscular, and intranasal. The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, compound according to the invention is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or non-aqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as pepper-mint, methyl salicylate, or orange flavoring.

Injectable compositions are typically based upon injectable sterile saline or phosphate buffered saline or other injectable carriers known in the art. As above mentioned, spiro derivatives of Formula (I) in such compositions is typically a minor component, frequently ranging between 0.05 to 10% by weight with the remainder being the injectable carrier and the like.

The above-described components for orally administered or injectable compositions are merely representative. Further materials as well as processing techniques and the like are set out in Part 5 of Remington's Pharmaceutical Sciences, 20th Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

Pharmaceutical formulations can be administered in the form of dosage units, which comprise a predetermined amount of active ingredient per dosage unit. Such a unit can comprise, for example, 0.5 mg to 1 g, preferably 1 mg to 700 mg, particularly preferably 5 mg to 100 mg, of a compound according to the invention, depending on the disease condition treated, the method of administration and the age, weight and condition of the patient, or pharmaceutical formulations can be administered in the form of dosage units which comprise a predetermined amount of active ingredient per dosage unit. Preferred dosage unit formulations are those which comprise a daily dose or part-dose, as indicated above, or a corresponding fraction thereof of an active ingredient. Furthermore, pharmaceutical formulations of this type can be prepared using a process, which is generally known in the pharmaceutical art.

In a seventh aspect, the invention provides a method of synthesis of a compound according to Formulae (I) and related formulae.

The spiro derivatives exemplified in this invention may be prepared from readily available starting materials using the following general methods and procedures. It will be appreciated that where typical or preferred experimental conditions (i.e. reaction temperatures, time, moles of reagents, solvents etc.) are given, other experimental conditions can also be used unless otherwise stated. Optimum reaction conditions may vary with the particular reactants or solvents used, but such conditions can be determined by the person skilled in the art, using routine optimization procedures.

In a height aspect, the present invention relates to a kit separate packs of
(a) an effective amount of a compound according to formula (I) and/or related formulae and/or pharmaceutically usable derivatives, tautomers, salts, solvates and stereoisomers thereof, including mixtures thereof in all ratios,
   and
(b) an effective amount of a further medicament active ingredient
In one embodiment, the separate packs consist of distinct containers or vessels, each of them containing either the effective amount of formula (I) or an effective amount of a further active ingredient.

In a second embodiment the kit may also comprise a third vessel containing an adjuvant or a diluent. In a third embodiment, the kit is used to prepare the pharmaceutical composition of the present invention.

In a ninth aspect, the present invention relates to a commercial package consisting of an effective amount of a compound according to formula (I), and/or pharmaceutically usable derivatives, tautomers, salts, solvates and stereoisomers thereof, including mixtures thereof in all ratios, together with instructions for the use thereof in treatment of allergic diseases and inflammatory dermatoses.

The following abbreviations refer to the abbreviations used below:

min (minute), hr (hour), g (gram), MHz (Megahertz), ml (milliliter), mmol (millimole), mM (millimolar), RT (room temperature), AcNH2 (Acetamide), AcOH (Acetic acid), ATP (Adenoside Triphosphate), BSA (Bovine Serum Albumin), Bu4NOH (Tetrabutylammonium hydroxide), CDI (1,1′-Carbonyldiimidazole), DBU (1,8-Dizabicyclo[5.4.0]undec-7-ene), DCM (Dichloromethane), DIPEA (di-isopropyl ethylamine), DMAP (4-Dimethylaminopyridine), DMSO (Dimethyl Sulfoxide), DMF (N,N-Dimethylformamide), CH3NO2 (Nitromethane), CsCO3 (Cesium carbonate), cHex (Cyclohexanes), Et3N (Triethylamine), EtOAc (Ethyl acetate), EtOH (Ethanol), HCl (hydrogen chloride), K2CO3 (Potassium Carbonate), NaI (Sodium Iodine), KCN, (Potassium cyanide), MeOH (Methanol), MgSO4 (Magnesium sulfate), NH3 (ammonia), NaH (Sodium hydride), NaHCO3 (Sodium bicarbonate), NH4Cl (Ammonium chloride), NH4(CO3)2 (ammonium carbonate), TEA (Triethyl amine), TFA (Trifluoroacetic acid), THF (Tetrahydrofuran), tBuOK (Potassium tert-butoxide), PdCl2 (Palladium dichloride), PetEther (Petroleum ether), PtO2 (Platinium oxide), TBME (tert-Butyl Methyl Ether), TMSI (Trimethylsilyl iodide), Zn (Zinc powder), rt (room temperature). HPLC (High Performance Liquid Chromatography), FC (Flash Chromatography on silica gel), MS (Mass Spectrometry), NMR (Nuclear Magnetic Resonance), PBS (Phosphate Buffered Saline), SPA (Scintillation Proximity Assay), TLC (Thin Layer Chromatography), UV (Ultraviolet).

If the above set of general synthetic methods is not applicable to obtain compounds according to Formula (I) and/or necessary intermediates for the synthesis of compounds of Formula (I), suitable methods of preparation known by a person skilled in the art should be used. In general, the synthesis pathways for any individual compound of Formula (I) will depend on the specific substituents of each molecule and upon the ready availability of intermediates necessary; again such factors being appreciated by those of ordinary skill in the art. For all the protection and deprotection methods, see Philip J. Kocienski, in “Protecting Groups”, Georg Thieme Verlag Stuttgart, New York, 1994 and, Theodora W. Greene and Peter G. M. Wuts in “Protective Groups in Organic Synthesis”, Wiley Interscience, 3rd Edition 1999.

Compounds of this invention can be isolated in association with solvent molecules by crystallization from evaporation of an appropriate solvent. The pharmaceutically acceptable acid addition salts of the compounds of Formula (I), which contain a basic center, may be prepared in a conventional manner. For example, a solution of the free base may be treated with a suitable acid, either neat or in a suitable solution, and the resulting salt isolated either by filtration or by evaporation under vacuum of the reaction solvent. Pharmaceutically acceptable base addition salts may be obtained in an analogous manner by treating a solution of compound of Formula (I) with a suitable base. Both types of salts may be formed or interconverted using ion-exchange resin techniques.

In the following the present invention shall be illustrated by means of some examples, which are not construed to be viewed as limiting the scope of the invention.

### General:

The HPLC data provided in the examples described below were obtained as followed.

Condition A: Column Waters Xbridge™ C8 50 mm×4.6 mm at a flow of 2 mL/min; 8 min gradient from 0.1% TFA in H2O to 0.07% TFA in CH3CN.

Condition B (chiral HPLC): Column Chiralcel OJ-H, 250×4.6 mm at a flow of 1 mL/min; eluant 0.1% formic acid in methanol.

UV detection (maxplot) for all conditions.

The MS data provided in the examples described below were obtained as followed: Mass spectrum:

LC/MS Waters ZMD (ESI)

The NMR data provided in the examples described below were obtained as followed: 1H-NMR: Bruker DPX-300 MHz.

Preparative HPLC purifications were performed with a mass directed autopurification Fractionlynx from Waters equipped with a Sunfire Prep C18 OBD column 19×100 mm 5 min, unless otherwise reported. All HPLC purifications were performed with a gradient of ACN/H2O or ACN/H2O/HCOOH (0.1%).

Optical rotations were measured using an APP220 Bellingham Stanley Ltd Polarimeter, with a cell length of 1 dm, using a sodium “D” light source (589 nm) at 25° C. αD values were calculated using the formula αD=100*α/(1*c), whereas “α” is the measured rotation (in degrees), “1” the length of the cell (in dm) and “c” the concentration of the test compound (in g/100 mL). αD values are expressed in 10⁻¹*deg*cm²*g⁻¹.

The compounds of invention have been named according to the standards used in the program “ACD/Name Batch” from Advanced Chemistry Development Inc., ACD/Labs (7.00 Release). Product version: 7.10, build: 15 Sep. 2003

### Intermediate 1: (+)-[5′-Chloro-1-(5-chloro-2-fluorobenzyl)-2,2′,5-trioxospiro[imidazolidine-4,3′-indol]-1′(2′H)-yl]acetic acid

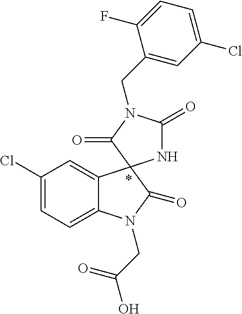

The two enantiomers of [5′-Chloro-1-(5-chloro-2-fluorobenzyl)-2,2′,5-trioxospiro[imidazolidine-4,3′-indol]-1′(2′H)-yl]acetic acid, prepared as described in WO2006125784 (Example 109), were separated by chromatography on a Daicel OJ 20 microns stationary phase, using methanol (containing 0.05% TFA) as eluant, with a flow of 300 mL/min The compound was fed at a concentration of 20 mg/mL. Each run lasted 12 minutes, and the retention times for the two enantiomers were respectively 6.57 min and 9.9 min (selectivity 1.6).

Of the two enantiomers obtained, the first eluting enantiomer showed the better activity on DP2 and was used as starting material for the compounds of this patent (Examples 2-4) and designated as Intermediate 1.

The second eluted enantiomer may also be used as starting material.

¹H NMR (300 MHz, DMSO-d₆) δ[ppm] 13,30 (bs, 1H), 9.34 (s, 1H), 7.68 (d, J=2.0 Hz, 1H), 7.53 (d, J=2.2 Hz, J=8.5 Hz, 1H), 7.48-7.42 (m, 1H), 7.34-7.28 (m, 2H), 7.21 (d, J=8.5 Hz, 1H), 4.68 (s, 2H), 4.58 (d, J=17.9 Hz, 1H), 4.53 (d, J=17.9 Hz, 1H). MS (ESI−): 450.1. HPLC (Condition A): Rt 3.79 min (HPLC purity 99.1%). Chiral HPLC (Condition B) Rt 7.44 min (HPLC purity 99.1%). αD=+49.1±5.6 (c=0.71 g/100 mL, MeOH).

### Intermediate 2: (+)-tert-butyl[5′-chloro-1-(5-chloro-2-fluorobenzyl)-2,2′,5-trioxospiro[imidazolidine-4,3′-indol]-1′(2′H)-yl]acetate

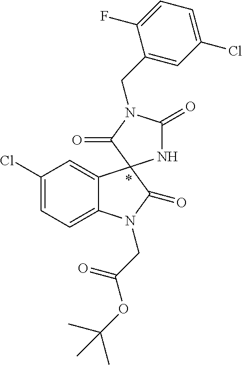

A solution of (+)-[5′-Chloro-1-(5-chloro-2-fluorobenzyl)-2,2′,5-trioxospiro[imidazolidine-4,3′-indol]-1′(2′H)-yl]acetic acid (Intermediate 1; 1.00 g; 2.21 mmol) in THF (10 ml) was treated with a solution of tert-butyl N,N′-diisopropylimidocarbamate (3.54 g; 17.7 mmol; prepared according to the general protocol described by Mathias, Synthesis, 1979, 561-576) in DCM (2 mL). The reaction mixture was stirred overnight at RT, then left to stand for 1 hour. The white solid was filtered off and washed with DCM, then the solvents were evaporated under vacuum. The residue was purified by flash column chromatography, eluting with cyclohexane containing increasing amounts of EtOAc to give the title compound as a white solid (683 mg).

Starting from the opposite enantiomer of intermediate 1 provides the opposite enantiomer of the intermediate 2.

¹H NMR (300 MHz, DMSO-d₆) δ[ppm] 9.33 (s, 1H), 7.68 (d, J=2.0 Hz, 1H), 7.54 (d, J=2.2 Hz, J=8.5 Hz, 1H), 7.47-7.42 (m, 1H), 7.34-7.28 (m, 2H), 7.18 (d, J=8.5 Hz, 1H), 4.68 (s, 2H), 4.56 (s, 2H), 1.40 (s, 9H). HPLC (Condition A): Rt 5.13 min (HPLC purity 99.1%). αD=+64.4±5.6 (c=0.71 g/100 mL, MeOH).

### Intermediate 3: tert-butyl (5′-chloro-3-methyl-2,2′,5-trioxospiro[imidazolidine-4,3′-indol]-1′(2′H)-yl)acetate

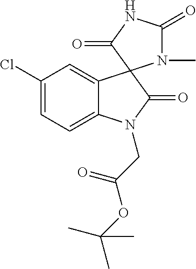

A cooled (0° C.) solution of tert-butyl (5′-chloro-2,2′,5-trioxospiro[imidazolidine-4,3′-indol]-1′(2′H)-yl)acetate (3.05 g; 8.34 mmol, prepared as described in WO2006125784, Intermediate 54) in THF (50.00 ml) was treated dropwise over 20 minutes with a solution of lithium bis(trimethylsilyl)amide (18.0 ml; 1.00 M; 18.0 mmol) in THF. The reaction solution was stirred for 1 h then iodomethane (0.60 ml; 9.6 mmol) was added dropwise. The reaction solution was then allowed to warm to rt. After stirring for 6 h the reaction mixture was carefully poured into 1M HCl and extracted with EtOAc, the organic phase was dried over MgSO₄, filtered and concentrated to give a residue which was then purified by flash column chromatography, eluting with cyclohexane containing increasing amounts of EtOAc to give the title compound as a yellow solid.

¹H NMR (300 MHz, DMSO-d₆) δ[ppm] 11.63 (br s, 1H), 7.75 (d, J=2.2 Hz, 1H), 7.55 (dd, J=8.5, 2.2 Hz, 1H), 7.22 (d, J=8.5 Hz, 1H), 4.66-4.48 (m, 2H), 2.57 (s, 3H), 1.39 (s, 9H). MS (ESI−): 378.3. HPLC (Condition A): Rt 3.73 min (HPLC purity 78.8%).

### EXAMPLES

### Example 1

### [5′-chloro-1-(5-chloro-2-fluorobenzyl)-3-methyl-2,2′,5-trioxospiro[imidazolidine-4,3′-indol]-1′(2′H)-yl]acetic acid

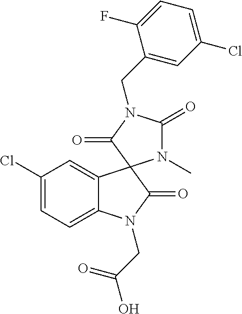

### Step 1: tert-butyl[5′-chloro-1-(5-chloro-2-fluorobenzyl)-3-methyl-2,2′,5-trioxospiro[imidazolidine-4,3′-indol]-1′(2′H)-yl]acetate

A solution of tert-butyl[5′-chloro-1-(5-chloro-2-fluorobenzyl)-2,2′,5-trioxospiro[imidazolidine-4,3′-indol]-1′(2′H)-yl]acetate (326 mg; 0.64 mmol, prepared as described in WO2006125784, Intermediate 20) and K2CO3 (177 mg; 1.28 mmol) in DMF (5 ml) was treated with iodomethane (120 μl; 1.92 mmol). The reaction mixture was stirred under nitrogen atmosphere for 16 h, then the reaction mixture was diluted with water and extracted with EtOAc twice. The combined organic phases were then washed three times with brine, dried over magnesium sulfate, filtered and concentrated under vacuum to give a residue which was purified by flash column chromatography, eluting with cyclohexane containing increasing amounts of EtOAc to give the title compound as a white solid.

¹H NMR (300 MHz, DMSO-d₆) δ[ppm] 7.43 (d, J=1.7 Hz, 1H), 7.42 (dd, J=2.3, 8.5 Hz, 1H), 7.32 (m, 1H), 7.29 (m, 3H), 4.69 (s, 2H), 4.59 (m, 2H), 2.64 (s, 3H), 1.38 (s, 9H). MS (ESI+) 539.1 (M+NH4+).

### Step 2: [5′-chloro-1-(5-chloro-2-fluorobenzyl)-3-methyl-2,2′,5-trioxospiro[imidazolidine-4,3′-indol]-1′(2′H)-yl]acetic acid

A solution of tert-butyl[5′-chloro-1-(5-chloro-2-fluorobenzyl)-3-methyl-2,2′,5-trioxospiro[imidazolidine-4,3′-indol]-1′(2′H)-yl]acetate (168 mg; 0.32 mmol) in DCM (4 ml) was treated with TFA (0.5 mL). The reaction mixture was stirred at RT for 4 h.

The solvents were removed under vacuum and the mixture was purified by flash column chromatography, eluting with DCM containing increasing amounts of MeOH to give the title compound as a white solid (127 mg, 85%).

¹H NMR (300 MHz, CDCl₃) δ[ppm] 7.29-7.26 (m, 2H), 7.17 (m, 1H), 7.09 (d, J=1.9 Hz, 1H), 6.94 (t, J=8.8 Hz, 1H), 6.81 (d, J=8.6 Hz, 1H), 4.71 (s, 2H), 4.34 (d, J=18.3 Hz, 1H), 4.20 (d, J=18.3 Hz, 1H), 2.63 (s, 3H). MS (ESI−): 464.1. HPLC (Condition A): Rt 4.72 min (HPLC purity 98.1%).

### Example 2

### (+)-[5′-chloro-1-(5-chloro-2-fluorobenzyl)-3-methyl-2,2′,5-trioxospiro[imidazolidine-4,3′-indol]-1′(2′H)-yl]acetic acid

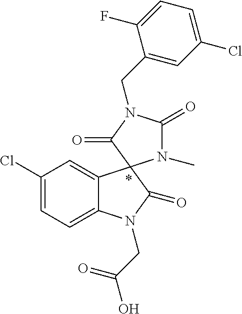

### Step 1: (+)-tert-butyl[5′-chloro-1-(5-chloro-2-fluorobenzyl)-3-methyl-2,2′,5-trioxospiro[imidazolidine-4,3′-indol]-1′(2′H)-yl]acetate

A solution of (+)-tert-butyl[5′-chloro-1-(5-chloro-2-fluorobenzyl)-2,2′,5-trioxospiro[imidazolidine-4,3′-indol]-1′(2′H)-yl]acetate (Intermediate 2; 2500 mg; 0.91 mmol), iodomethane (63 μl; 1.0 mmol) and K2CO3 (253 mg; 1.83 mmol) in DMF (10 mL) was stirred for 3 h. The reaction mixture was diluted with water and extracted with EtOAc three times. The combined organic phases were then washed with brine, dried over magnesium sulfate, filtered and concentrated to give the Title compound as a white foam.

MS (ESI+): 539.4. HPLC (Condition A): Rt 5.79 min (HPLC purity 99.7%).

### Step 2: (+)-[5′-chloro-1-(5-chloro-2-fluorobenzyl)-3-methyl-2,2′,5-trioxospiro[imidazolidine-4,3′-indol]-1′(2′H)-yl]acetic acid

A solution of (+)-tert-butyl[5′-chloro-1-(5-chloro-2-fluorobenzyl)-3-methyl-2,2′,5-trioxospiro[imidazolidine-4,3′-indol]-1′(2′H)-yl]acetate (422 mg; 0.81 mmol) in DCM (15 ml) and TFA (3 ml) was stirred under nitrogen atmosphere for 3 hours.

The solvents were removed under vacuum, the residue was redissolved in DCM and washed with water then brine. The organic phase was dried over MgSO₄, filtered and concentrated.

The oily solid was redissolved in EtOAc and precipitated by addition of cyclohexane. The solid was filtered and dried under vacuum, then redissolved in DCM. The solvent was removed under vacuum to give the Title compound as a white powder.

¹H NMR (300 MHz, DMSO-d₆) δ[ppm] 13.4 (brs, 1H), 7.77 (d, J=2.2 Hz, 1H), 7.56 (dd, J=2.5, 8.8 Hz, 1H), 7.41 (m, 1H), 7.32 (m, 2H), 7.27 (d, J=8.5 Hz, 1H), 4.69 (s, 2H), 4.58 (m, 2H), 2.63 (s, 3H). MS (ESI−): 464.1. HPLC (Condition A): Rt 4.30 min (HPLC purity 100%). Chiral HPLC (Condition B): Rt 5.67 min (HPLC purity 99.7%). CHN analysis: [C20H14N3O5Cl2F-0.5 H2O] Calculated: C 50.54%,H 3.18%,N 8.84%. Found: C 50.56%,H 2.94%,N 8.69%. αD=+152.6±23.5 (c=0.71 g/100 mL, MeOH).

Starting from the opposite enantiomer of intermediate 2 provides the opposite enantiomer.

### Example 3

### [(+)-5′-chloro-1-(5-chloro-2-fluorobenzyl)-3-ethyl-2,2′,5-trioxospiro[imidazolidine-4,3′-indol]-1′(2′H)-yl]acetic acid

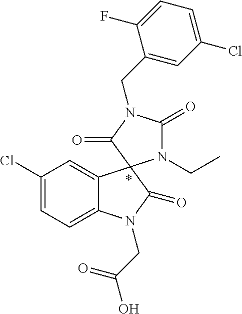

Following the two-steps general method as outlined in Example 1, starting from (+)-tert-butyl [5′-chloro-1-(5-chloro-2-fluorobenzyl)-2,2′,5-trioxospiro[imidazolidine-4,3′-indol]-1′(2H)-yl]acetate (Intermediate 2) and ethyl iodide (Fluka) the title compound was obtained as a white solid.

¹H NMR (300 MHz, DMSO-d₆) δ[ppm] 13.4 (brs, 1H), 7.80 (d, J=2.2 Hz, 1H), 7.58 (dd, J=2.2, 8.5 Hz, 1H), 7.45 (m, 1H), 7.34-7.27 (m, 3H), 4.71 (s, 2H), 4.63 (d, J=17.9 Hz, 1H), 4.58 (d, J=17.9 Hz, 1H), 3.30 (m, 1H), 3.13 (m, 1H), 0.91 (t, J=7.2 Hz, 3H). MS (ESI−): 478.2. HPLC (Condition A): Rt 4.24 min (HPLC purity 95.8%). αD=+97.1±19.4 (C=0.20 g/100 ml, MeOH)

Starting from the opposite enantiomer of intermediate 2 provides the opposite enantiomer.

### Example 4

### [((+)-5′-chloro-1-(5-chloro-2-fluorobenzyl)-2,2′,5-trioxo-3-propylspiro[imidazolidine-4,3′-indol]-1′(2′H)-yl]acetic acid

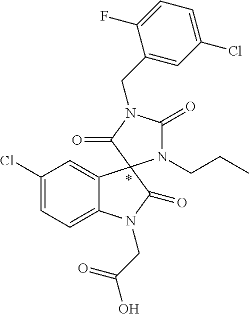

Following the two-steps general method as outlined in Example 1, starting from (+)-tert-butyl [5′-chloro-1-(5-chloro-2-fluorobenzyl)-2,2′,5-trioxospiro[imidazolidine-4,3′-indol]-1′(2′H)-yl]acetate (Intermediate 2) and propyl iodide (Merck Kgaa) the title compound was obtained as a white solid.

¹H NMR (300 MHz, DMSO-d₆) δ[ppm] 13.4 (brs, 1H), 7.78 (d, J=2.2 Hz, 1H), 7.57 (dd, J=2.2, 8.5 Hz, 1H), 7.45 (m, 1H), 7.34-7.26 (m, 3H), 4.72 (s, 2H), 4.60 (d, J=17.9 Hz, 1H), 4.57 (d, J=17.9 Hz, 1H), 3.20 (m, 1H), 3.04 (m, 1H), 1.28 (m, 2H), 0.73 (t, J=7.3 Hz, 3H). MS (ESI−): 492.3. HPLC (Condition A): Rt 4.46 min (HPLC purity 95.8%). αD=+48.2±19.3 (C=0.20 g/100 ml, MeOH).

Starting from the opposite enantiomer of intermediate 2 provides the opposite enantiomer.

### Example 5

### [5′-chloro-1-[(2-isopropyl-1,3-thiazol-4-yl)methyl]-3-methyl-2,2′,5-trioxospiro[imidazolidine-4,3′-indol]-1′(2′H)-yl]acetic acid

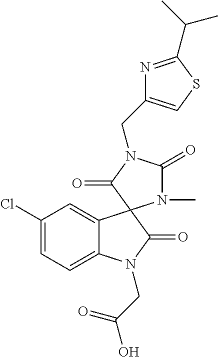

### Step 1: tert-butyl[5′-chloro-1-[(2-isopropyl-1,3-thiazol-4-yl)methyl]-3-methyl-2,2′,5-trioxospiro[imidazolidine-4,3′-indol]-1′(2′H)-yl]acetate

A solution of 4-(chloromethyl)-2-isopropylthiazole (Fluorochem; 97 mg; 0.55 mmol) in DMF (0.50 ml) was treated with a suspension of tert-butyl (5′-chloro-3-methyl-2,2′,5-trioxospiro[imidazolidine-4,3′-indol]-1′(2′H)-yl)acetate (Intermediate 3; 95 mg; 0.25 mmol), sodium hydrogen carbonate (94 mg; 1.1 mmol) and potassium iodide (10 mg; 0.06 mmol) in DMF (3.00 ml). The reaction mixture was heated at 80° C. for 16 h then cooled and concentrated under vacuum. The residue was dissolved in EtOAc and washed with a sat. NH4Cl solution, then the organic phase was dried on MgSO4, filtered and concentrated to give a residue which was purified by flash column chromatography, eluting with cyclohexane containing increasing amounts of EtOAc to give the title compound as a white solid.

¹H NMR (300 MHz, DMSO-d₆) δ[ppm] 7.68 (d, J=2.2 Hz, 1H), 7.60 (dd, J=8.5, 2.2 Hz, 1H), 7.30-7.25 (m, 2H), 4.73 (s, 2H), 4.63 (d, J=17.9 Hz, 1H), 4.59 (d, J=17.9 Hz, 1H), 3.25 (m, 1H), 2.67 (s, 3H), 1.39 (s, 9H), 1.33 (d, J=6.9 Hz, 3H), 1.31 (d, J=6.9 Hz, 3H). MS (ESI+): 519.2. HPLC (Condition A): Rt 5.02 min (HPLC purity 96.7%).

### Step 2: [5′-chloro-1-[(2-isopropyl-1,3-thiazol-4-yl)methyl]-3-methyl-2,2′,5-trioxospiro[imidazolidine-4,3′-indol]-1′(2′H)-yl]acetic acid

A solution of tert-butyl[5′-chloro-1-[(2-isopropyl-1,3-thiazol-4-yl)methyl]-3-methyl-2,2′,5-trioxospiro[imidazolidine-4,3′-indol]-1′(2′H)-yl]acetate (41 mg; 0.08 mmol) in HCl in Dioxane (4 N, 5 ml) was stirred for 16 h then concentrated. The residue was redissolved in a mixture of DCM/Et2O and concentrated then dried under vacuum to give a yellow solid (39 mg, quant.)

¹H NMR (300 MHz, DMSO-d₆) δ[ppm] 13.3 (br s, 1H), 7.68 (d, J=2.2 Hz, 1H), 7.58 (dd, J=8.5, 2.2 Hz, 1H), 7.31 (d, J=8.5 Hz, 1H), 7.28 (s, 1H), 4.73 (s, 2H), 4.63 (d, J=17.8 Hz, 1H), 4.59 (d, J=17.8 Hz, 1H), 3.27 (sep, J=6.9 Hz, 1H), 2.66 (s, 3H), 1.32 (d, J=6.9 Hz, 3H), 1.31 (d, J=6.9 Hz, 3H). MS (ESI−): 461.1. HPLC (Condition A): Rt 3.80 min (HPLC purity 93.8%).

### Example 6

### [5′-chloro-1-[(1,3-diphenyl-1H-pyrazol-4-yl)methyl]-3-methyl-2,2′,5-trioxospiro[imidazolidine-4,3′-indol]1′(2′H)-yl]acetic acid

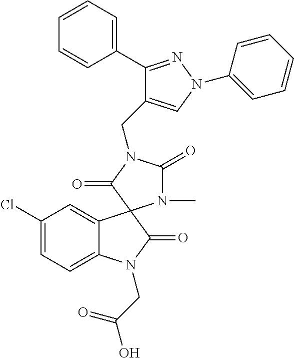

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 4-(chloromethyl)-1,3-diphenyl-1H-pyrazole (Enamine) the title compound was obtained as a white solid

¹H NMR (300 MHz, DMSO-d₆) δ[ppm] 8.36 (s, 1H), 7.85 (d, J=7.9 Hz, 2H), 7.77 (d, J=7.2 Hz, 2H), 7.66 (s, 1H), 7.60-7.24 (m, 8H), 4.90-4.70 (m, 2H), 4.69-4.50 (m, 2H), 2.66 (s, 3H). MS (ESI−): 554.2. HPLC (Condition A): Rt 4.61 min (HPLC purity 97.8%).

### Example 7

### [5′-chloro-3-methyl-1-[(5-methyl-3-phenylisoxazol-4-yl)methyl]-2,2′,5-trioxospiro[imidazolidine-4,3′-indol]-1′(2′H)-yl]acetic acid

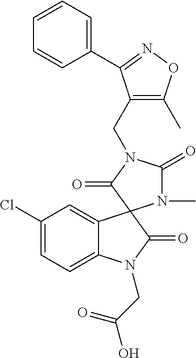

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 4-(bromomethyl)-5-methyl-3-phenylisoxazole (ABCR) the title compound was obtained as a white solid.

¹H NMR (300 MHz, DMSO-d₆) δ[ppm] 13.3 (br s, 1H), 7.69-7.62 (m, 2H), 7.57-7.42 (m, 5H), 7.25 (d, J=8.4 Hz, 1H), 4.66-4.44 (m, 4H), 2.57 (s, 3H), 2.46 (s, 3H). MS (ESI−): 493.3. HPLC (Condition A): Rt 3.92 min (HPLC purity 88.4%).

### Example 8

### [5′-chloro-3-methyl-2,2′,5-trioxo-1-[(2-phenyl-1,3-thiazol-4-yl)methyl] Spiro[imidazolidine-4,3′-indol]-1′(2′H)-yl]acetic acid

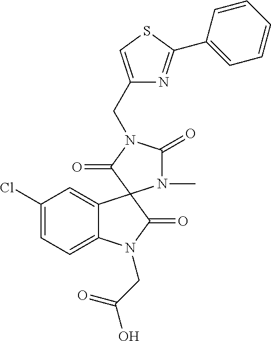

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 4-(chloromethyl)-2-phenyl-1,3-thiazole (ABCR) the title compound was obtained as a white solid.

¹H NMR (300 MHz, DMSO-d₆) δ[ppm] 13.4 (br s, 1H), 7.96-7.89 (m, 2H), 7.68 (d, J=2.2 Hz, 1H), 7.58 (dd, J=8.5, 2.2 Hz, 1H), 7.55-7.47 (m, 4H), 7.30 (d, J=8.5 Hz, 1H), 4.83 (s, 2H), 4.64 (d, J=17.9 Hz, 1H), 4.56 (d, J=17.9 Hz, 1H), 2.68 (s, 3H). MS (ESI−): 495.2. HPLC (Condition A): Rt 4.12 min (HPLC purity 97.1%).

### Example 9

### [5′-chloro-1-[(4-methoxy-3,5-dimethylpyridin-2-yl)methyl]-3-methyl-2,2′,5-trioxospiro[imidazolidine-4,3′-indol]-1′(2′H)-yl]acetic acid

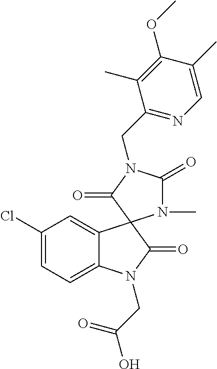

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 2-chloromethyl-4-methoxy-3,5-dimethylpyridine hydrochloride (Sigma-Aldrich) the title compound was obtained as a white solid.

¹H NMR (300 MHz, DMSO-d₆) δ[ppm] 13.4 (br s, 1H), 8.15 (s, 1H), 7.63-7.56 (m, 2H), 7.28 (d, J=8.4 Hz, 1H), 4.77 (s, 2H), 4.58 (d, J=17.9 Hz, 1H), 4.53 (d, J=17.9 Hz, 1H), 3.73 (s, 3H), 2.68 (s, 3H), 2.22 (s, 3H), 2.21, (s, 3H). MS (ESI−): 471.2. HPLC (Condition A): Rt 2.58 min (HPLC purity 96.3%).

### Example 10

### [5′-chloro-1-(2,5-difluorobenzyl)-3-methyl-2,2′,5-trioxospiro[imidazolidine-4,3′-indol]-1′(2′H)-yl]acetic acid

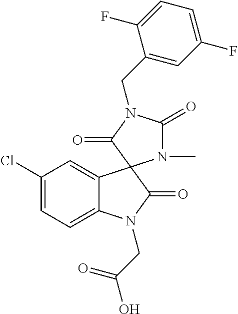

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 2,5-difluorobenzyl bromide (Sigma-Aldrich) the title compound was obtained as a white solid.

¹H NMR (300 MHz, DMSO-d₆) δ[ppm] 13.4 (br s, 1H), 7.79 (d, J=2.2 Hz, 1H), 7.58 (dd, J=8.5, 2.2 Hz, 1H), 7.37-7.18 (m, 3H), 7.09 (m, 1H), 4.72 (s, 2H), 4.62 (d, J=17.8 Hz, 1H), 4.56 (d, J=17.8 Hz, 1H), 2.65 (s, 3H). MS (ESI−): 448.2. HPLC (Condition A): Rt 3.90 min (HPLC purity 98.9%).

### Example 11

### [1-(1,3-benzothiazol-2-ylmethyl)-5′-chloro-3-methyl-2,2′,5-trioxospiro[imidazolidine-4,3′-indol]-1′(2′H)-yl]acetic acid

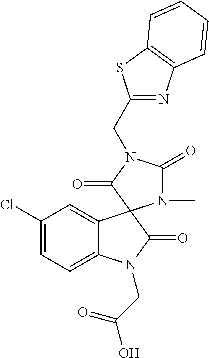

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 2-(bromomethyl)-1,3-benzothiazole (Acros) the title compound was obtained as a yellow solid.

¹H NMR (300 MHz, DMSO-d₆) δ[ppm] 13.40 (br s, 1H), 8.13 (d, J=7.3 Hz, 1H), 8.01 (d, J=7.7 Hz, 1H), 7.78 (d, J=2.2 Hz, 1H), 7.64-7.42 (m, 3H), 7.31 (d, J=8.5 Hz, 1H), 5.17 (s, 2H), 4.63 (d, J=17.7 Hz, 1H), 4.55 (d, J=17.7 Hz, 1H), 2.70 (s, 3H). MS (ESI−): 469.2. HPLC (Condition A): Rt 3.81 min (HPLC purity 96.5%).

### Example 12

### [5′-chloro-1-[2-(2-chlorophenyl)ethyl]-3-methyl-2,2′,5-trioxospiro[imidazolidine-4,3′-indol]-1′(2′H)-yl]acetic acid

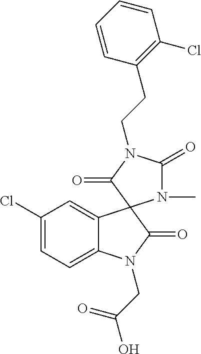

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 1-(2-bromo-ethyl)-2-chloro-benzene (Oakwood) the title compound was obtained as a brown solid.

¹H NMR (300 MHz, DMSO-d₆) δ[ppm] 13.30 (br s, 1H), 7.60-7.23 (m, 7H), 4.62 (d, J=17.8 Hz, 1H), 4.52 (d, J=17.8 Hz, 1H), 3.84-3.69 (m, 2H), 3.12-2.94 (m, 2H), 2.60 (s, 3H). MS (ESI−): 460.1. HPLC (Condition A): Rt 4.12 min (HPLC purity 92.1%).

### Example 13

### [15′-chloro-1-[2-(3-fluorophenyl)ethyl]-3-methyl-2,2′,5-trioxospiro[imidazolidine-4,3′-indol]-1′(2′H)-yl]acetic acid

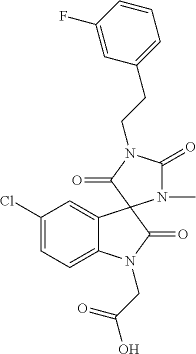

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 2-(3-fluorophenyl)ethyl bromide (ABCR) the title compound was obtained as a yellow solid.

¹H NMR (300 MHz, DMSO-d₆) δ[ppm] 13.3 (br s, 1H), 7.55 (dd, J=8.5, 2.2 Hz, 1H), 7.41-7.23 (m, 3H), 7.13-6.99 (m, 3H), 4.59 (d, J=17.9 Hz, 1H), 4.55 (d, J=17.9 Hz, 1H), 3.75 (t, J=7.0 Hz, 2H), 2.93 (t, J=7.0 Hz, 2H), 2.59 (s, 3H). MS (ESI−): 444.2. HPLC (Condition A): Rt 4.09 min (HPLC purity 94.8%).

### Example 14

### [15′-chloro-1-[2-(2-fluorophenyl)ethyl]-3-methyl-2,2′,5-trioxospiro[imidazolidine-4,3′-indol]-1′(2′H)-yl]acetic acid

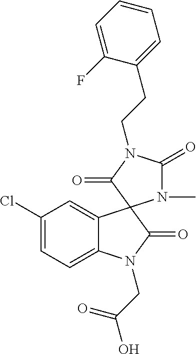

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 1-(2-bromo-ethyl)-2-fluoro-benzene (Oakwood) the title compound was obtained as a yellow solid.

¹H NMR (300 MHz, DMSO-d₆) δ[ppm] 13.3 (br s, 1H), 7.55 (dd, J=8.5, 2.1 Hz, 1H), 7.42 (d, J=2.1 Hz, 1H), 7.36-7.24 (m, 3H), 7.19-7.09 (m, 2H), 4.60 (d, J=17.9 Hz, 1H), 4.55 (d, J=17.9 Hz, 1H), 3.73 (t, J=6.8 Hz, 2H), 3.03-2.88 (m, 2H), 2.59 (s, 3H). MS (ESI−): 444.1. HPLC (Condition A): Rt 3.90 min (HPLC purity 97.1%).

### Example 15

### [15′-chloro-1-[2-(3-chlorophenyl)ethyl]-3-methyl-2,2′,5-trioxospiro[imidazolidine-4,3′-indol]-1′(2′H)-yl]acetic acid

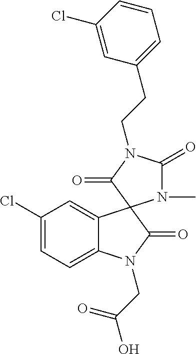

Following the two-steps general method as outlined in Example 5, starting from tert-butyl (5′-chloro-3-methyl-2,2′,5-trioxospiro[imidazolidine-4,3′-indol]-1′(2′H)-yl)acetate (Intermediate 3) and 1-(2-bromoethyl)-3-chlorobenzene (Oakwood) the title compound was obtained as a white foam.

¹H NMR (300 MHz, DMSO-d₆) δ[ppm] 13.3 (br s, 1H), 7.55 (dd, J=8.5, 2.2 Hz, 1H), 7.38 (d, J=2.2 Hz, 1H), 7.35-7.18 (m, 5H), 4.60 (d, J=17.9 Hz, 1H), 4.50 (d, J=17.9 Hz, 1H), 3.79-3.69 (m, 2H), 2.97-2.87 (m, 2H), 2.59 (s, 3H). MS (ESI−): 460.2. HPLC (Condition A): Rt 4.17 min (HPLC purity 94.5%).

### Example 16

### Preparation of hCRTH₂—CHO Expressing Cell Membranes

Adherent CHO cells expressing hCRTH2 (Euroscreen, Belgium) were cultured in 225 cm2 cell culture flasks (Corning, USA) in 30 ml of medium. After two rinses of phosphate buffered saline (PBS), cells were harvested in 10 ml of PBS containing 1 mM EDTA, centrifuged at 500×g for 5 min at 4° C. and frozen at −80° C. The pellet was re-suspended in 50 mM Tris-HCl, pH 7.4, 2 mM EDTA, 250 mM Sucrose, containing protease inhibitor cocktail tablets, (Complete EDTA-free, Roche, Germany) and incubated 30 min at 4° C. Cells were disrupted by nitrogen cavitation (Parr Instruments, USA) at 4° C. (800 p.s.i. for 30 min), and centrifuged at 500×g for 10 min at 4° C. Pellet containing nuclei and cellular debris was discarded and supernatant was centrifuged 60 min at 4° C. at 45000×g. Membrane pellet was re-suspended in storage buffer (10 mM HEPES/KOH pH 7.4, 1 mM EDTA, 250 mM sucrose, protease inhibitor cocktail tablets) using Dounce homogenization and frozen in liquid nitrogen, and stored at −80° C.

### Example 17

### Radioligand Binding Assay

The compounds of the present invention inhibit the binding of PGD2 to its receptor CRTH2. The inhibitory activity can be investigated by a radioligand binding Scintillation Proximity Assay (SPA) (Sawyer et al., Br. J. Pharmocol 2002, 137, 1163-72). The SPA radioligand binding assay was performed at room temperature in binding buffer (10 mM HEPES/KOH pH 7.4, 10 mM MnC12, with protease inhibitor cocktail tablets), containing 1.5 nM [3H]PGD2 (Perkin Elmer), 10-50 μg/ml of hCRTH₂—CHO cell membrane protein and 2 mg/ml of Wheat-germ agglutinin Scintillation Proximity Assay beads (RPNQ0001, GE-Healthcare) in a final volume of 100 μl in 96 well plates (Corning, USA). Non-specific binding was determined in the presence of 10 μM PGD2 (Cayman, USA). Competing Compounds of Formula (I) were diluted in dimethylsulphoxide so that the total volume of dimethylsulfoxide was kept constant at 1% dimethylsulphoxide (Me₂SO). Serial dilutions of 100 μM to 100 pM were prepared and 10 μl each of the compounds of Formula (I) stock solutions were added to the binding assay reagents and incubated for 90 min with agitation at room temperature. Binding activity was determined by using a 1450 Micro-beta scintillation counter (Wallac, UK).

In one embodiment, the compounds of Formula (I) of the present invention inhibit CRTH2 at a concentration of <5 μM. Preferably, the compounds of Formula (I) of the present invention inhibit CRTH2 at a concentration of <1 μM. most preferably, the compounds of Formula (I) of the present invention inhibit CRTH2 at a concentration of <0.1 μM.

### Results:

| | | IC50 |
|---|---|---|
| | | binding |
| Example | Formula | (μM) |
| 1 | 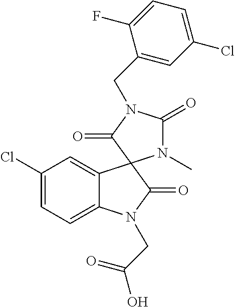 | 0.062 |
| 2 | 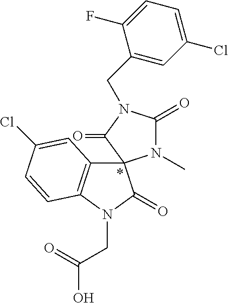 | 0.045 |
| 3 | 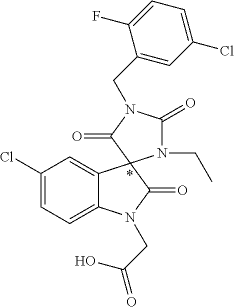 | 0.336 |
| 4 | 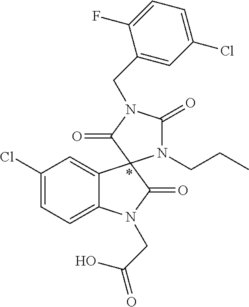 | 0.144 |
| 5 | 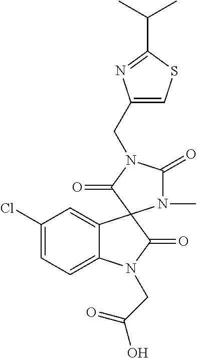 | 0.628 |
| 6 | 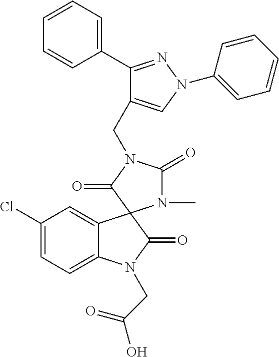 | 0.123 |
| 7 | 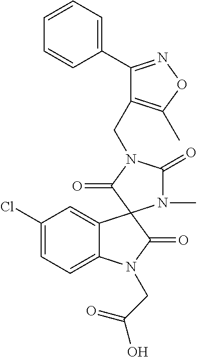 | 0.148 |
| 8 | 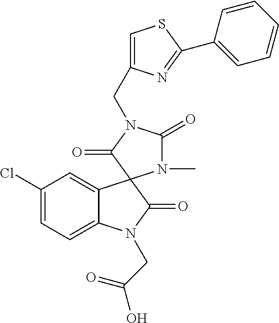 | 0.044 |
| 9 | 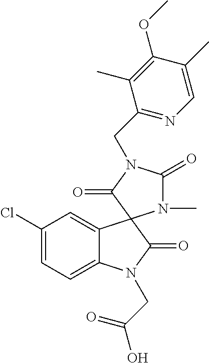 | 0.363 |
| 10 | 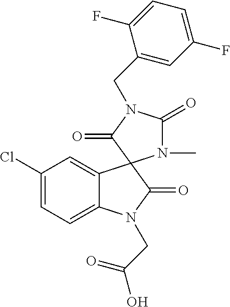 | 0.498 |
| 11 | 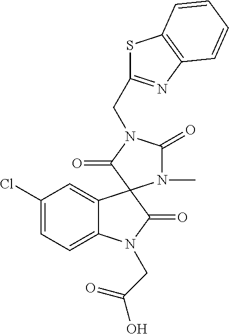 | 0.945 |
| 12 | 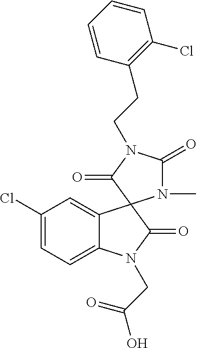 | 0.740 |
| 13 | 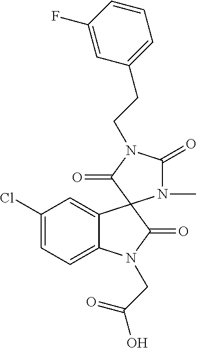 | 0.916 |
| 14 | 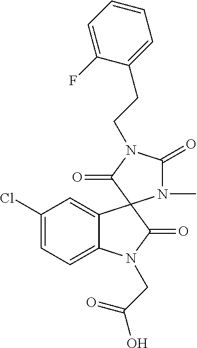 | 0.940 |
| 15 | 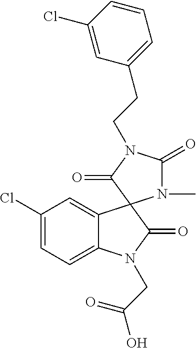 | 0.551 |

### Example 18

### PGD2-induced Eosinophil Cell Shape Assay in Human Whole Blood

The test compounds were diluted in dimethylsulphoxide so that the total volume of dimethylsulfoxide was kept constant at 2% dimethylsulphoxide (Me₂SO). Serial dilutions of 200 μM to 0.09 μM were prepared. Samples of 90 μl of human blood from healthy volunteers (Centre de Transfusion Sanguine de Genève) were pre-incubated in polypropylene Falcon tubes (BD 352063) for 20 minutes in a water bath at 37° C. with 10 μl of diluted compounds. For CRTH2 activation, 100 μl PGD2 (Cayman 12010) at 20 nM was added (10 nM final) to each tube and cells were maintained at 37° C. For negative control cells were treated with PBS. After 10 minutes, cell activation was stopped with 120 μl Formaldehyde 10% (4% final, Fluka 41650) and cells were rested for 10 minutes at room temperature. Fixed cells were transferred into polypropylene tubes and then treated for 1 hour in a water bath at 37° C. with 2 ml of Triton—Surfact-Amps X-100 (Pierce 28314) at 0.166% (0.13% Triton final). After several washes with PBS (red cells lysed progressively during washes, two washes are necessary), cells were analyzed by flow cytometry on a FACSCalibur.

### Example 19

### In Vivo Pharmacokinetic Evaluation in Rat and Mouse

In order to study the pharmacokinetic (PK) profile of test compounds in vivo, Sprague Dawley male rats or C57BL/6 female mice were dosed intravenously or after oral gavage. For both species, test compounds were dosed in solution at 1 mg/kg for i.v. route (10% ethanol, 10% N,N-dimethylacetamide, 30% propylene glycol, 50% water, v/v) and in suspension at 5 mg/kg (0.5% carboxymethylcellulose suspension, containing 0.25% Tween 20 in water) for oral gavage. PK profile in rat was obtained from 3 animals per dosing route and mouse PK profile was determined from 3 animals for each time points. The volume of administration was 2 mL/kg for i.v. dosing in both species and either 5 mL/kg (rat) or 10 mL/kg (mouse) for oral gavage. Blood samples (100 □L/time point) were collected at 0.083 (5 min), 0.25, 0.5, 1, 4, 7 and 24 hours post-dose for i.v. dosing, and at 0.5, 1, 4, 7 and 24 h for oral dosing, into heparin-Li+ containing tubes. For rats, all blood samples were collected trough a catheter in the carotid artery (placed in the artery the day before the experiment), under light isoflurane anesthesia, and stored on ice until centrifugation and plasma isolation. For mouse, blood samples were collected from intracardiac puncture at sacrifice at each time point and processed as described above for the rat. Plasma samples were stored frozen until analysis (−20° C. to −70° C.). For bioanalysis, samples were processed by protein precipitation (acetonitrile, formic acid 0.1%, addition of 3 volumes) after addition of one internal standard and analysed using a sensitive and selective LC/MS/MS method. An aliquot of the resulting supernatant was subject to LC/MS/MS analysis using a reverse phase column (Waters Xterra, C8, (3.5 μm particle size, 2.1×50 mm) and a short gradient (1 min) from (Solvent A) 85% water, 15% acetonitrile and 0.1% formic acid to (Solvent B) 90% acetonitrile, 10% water and 0.1% formic acid followed by isocratic conditions of Solvent B for 3.5 min at 0.4 mL/min. Column effluent was monitored using a Sciex API 4000 triple quadrupole mass spectrometer with a Turbo V electrospray ion source. Unknown concentrations of test compounds were determined using a calibration curve ranging from 1 to 3000 ng/mL.

### Example 20

### OVA-Induced Lung Eosinophilia in Mice

BALB/c mice (6-8 weeks old) were immunized with ovalbumin (10 μg i.p) on day 0 and 7. In order to elicit a local inflammatory response in the lung, mice were challenged between day's 15-17 with a nebulised solution of ovalbumin (10 μg/ml; De Vilibiss Ultraneb 2000, once daily for 30 min during the 3 days). On each separate day between 15 and 17 each animal received via oral gavage the test compound, at t−1 h and t+7 h with respect to OVA exposure at t=0 h. Eight hours after the final OVA challenge, bronchoalveolar lavage (BAL) was then carried out. Total cell numbers in the BAL fluid samples were measured using a haemocytometer. Cytospin smears of the BAL fluid samples were prepared by centrifugation at 1200 rpm for 2 min at room temperature and stained using a DiffQuik stain system (Dade Behring) for differential cell counts.

Compound of Example 2 showed activity in a model of OVA-induced eosinophil recruitment in lung when dosed by the oral route (79% inhibition at a dose of 30 mg/Kg).

The compound of Example 2 shows a significant improvement in the oral bioavailability in both mouse and rat. Oral bioavailability is a highly desirable feature in a DP2 antagonist as it allows the drug to be delivered by oral route (as solution, suspension, pills, tablets, capsules or the like)

| | 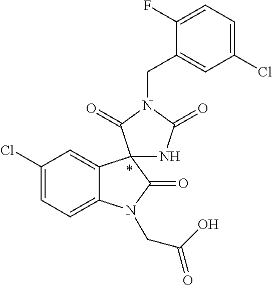 | 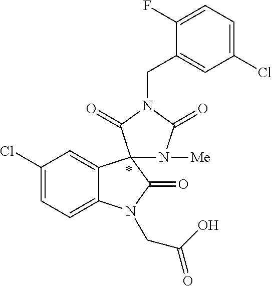 |
|---|---|---|
| | (+)-Enantiomer | Example 2 |
| Binding IC₅₀ | 21 nM | 68 nM |
| Whole Blood IC₅₀ | 50 nM | 150 nM |
| F_{z} in mice | 6% | 39% |
| CMax po in mice (5 mg/Kg) | 113 ng/mL | 691 ng/mL |
| F_{z} in rats | 5.3% | 37% |
| CMax po in rats (5 mg/Kg) | 151 ng/mL | 489 ng/mL |

### Example 21

### Preparation of a Pharmaceutical Formulation

### Formulation 1—Tablets

A compound of formula (I) is admixed as a dry powder with a dry gelatin binder in an approximate 1:2 weight ratio. A minor amount of magnesium stearate is added as a lubricant. The mixture is formed into 240-270 mg tablets (80-90 mg of active compound according to the invention per tablet) in a tablet press.

### Formulation 2—Capsules

A compound of formula (I) is admixed as a dry powder with a starch diluent in an approximate 1:1 weight ratio. The mixture is filled into 250 mg capsules (125 mg of active compound according to the invention per capsule).

### Formulation 3—Liquid

A compound of formula (I) (1250 mg), sucrose (1.75 g) and xanthan gum (4 mg) are blended, passed through a No. 10 mesh U.S. sieve, and then mixed with a previously prepared solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

### Formulation 4—Tablets

A compound of formula (I) is admixed as a dry powder with a dry gelatin binder in an approximate 1:2 weight ratio. A minor amount of magnesium stearate is added as a lubricant. The mixture is formed into 450-900 mg tablets (150-300 mg of active compound according to the invention) in a tablet press.

### Formulation 5—Injection

A compound of formula (I) is dissolved in a buffered sterile saline injectable aqueous medium to a concentration of approximately 5 mg/mL.

1-15. (canceled)

16. A compound of formula (I) 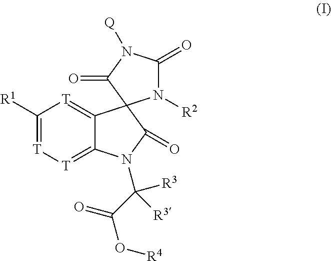
wherein:
R¹ is H, Hal, A, CN, OA, CF₃, or OCF₃,
R² is A,
R³, R³′ are independently from one another H or A,
R⁴ is H or A;
Q is A, —(CH₂)ₙ—Ar, —(CH₂)ₙHet, —(CH₂)ₚ—(CHR¹¹)_{q}—(CH₂)ᵣ—Ar, or —(CH₂)ₚ—(CHR¹¹)_{q}—(CH₂)ᵣ—Het,
p and r are independently from one another 0, 1, 2, 3 or 4,
q is 1 or 2,
R¹¹ denotes H, A, CN, OR⁶, Hal, Ar, or Het,
n is 1, 2, 3, or 4,
T is CR⁵ or N,
R⁵ is H, Hal, A, CN, OA, CF₃, or OCF₃,
A is branched or linear alkyl having 1 to 12 C-atoms, wherein one or more H-atoms may be replaced by Hal, OR⁶, CN, N(R⁶)₂, cycloalkyl Ar or Het, and wherein one or more CH₂-groups may be replaced by O, NR⁶CON(R)₂ or S and/or by CH═CH— or —C≡C— groups, or denotes cycloalkylen or cycloalkylalkylen having 3 to 7 ring C atoms,
Hal is F, CI, Br or I,
Ar denotes a monocyclic or bicyclic, unsaturated or aromatic carbocyclic ring having 6 to 14 carbon atoms which may be unsubstituted or monosubstituted, disubstituted or tri substituted by Hal, A, CH₂OA, —CH₂OR⁶, OR⁶, CF₃, OCF₃, N(R⁶)₂, NO₂, CN, NR⁶COA, NR⁶SO₂A, COR⁶, SO₂N(R⁶)₂, SOA, SO₂A, Het, or Ar′,
Ar′ denotes a monocyclic or bicyclic, unsaturated or aromatic carbocyclic ring having 6 to 14 carbon atoms which may be unsubstituted or monosubstituted, disubstituted or trisubstituted by Hal, A, —CH₂OA, —CH₂OR⁶, —OR⁶, —CF₃, or —OCF₃,
Het denotes a monocyclic or bicyclic, saturated, unsaturated or aromatic heterocyclic ring, having 1 to 4 N, O and/or S atoms, which may be unsubstituted or monosubstituted, disubstituted or trisubstituted by Hal, A, CH₂OA, OR⁶, CF₃, OCF₃, N(R⁶)₂, NO₂, CN, NR⁶COA, NR⁶SO₂A, COR⁶, SO₂N(R⁶)₂, SOA, SO₂A, or Ar,
R⁶ is H or A,
or derivatives, enantiomers, diastereoisomers, tautomers, salts, solvates and mixtures thereof.

17. The compound according to claim 16 wherein the compound is a pure enantiomer or a enantiomerically enriched mixture of enantiomers.

18. The compound according to claim 17, wherein said compound is an enantiomer of Formula I′a, I′b or I′c: 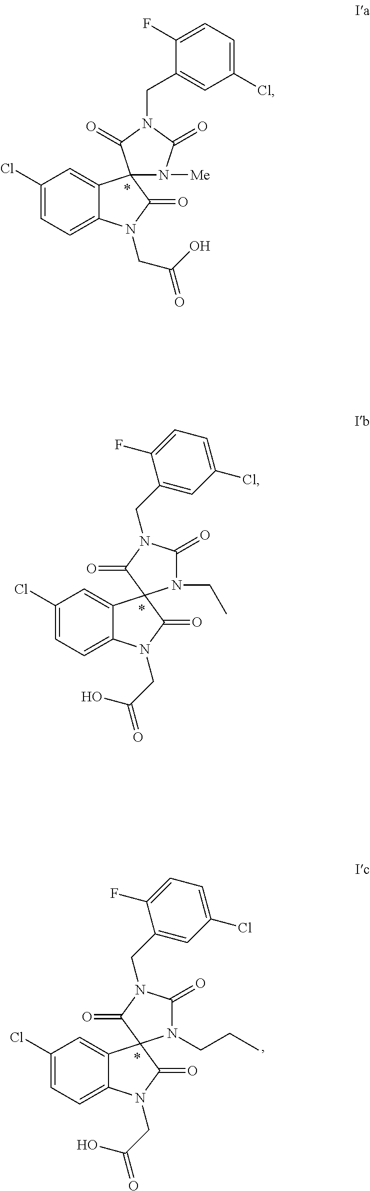
wherein the optical rotatory power is positive in MeOH.

19. The compound according to claim 16, wherein Q is selected from an alkyl having 1 to 6 carbon atoms or from the following groups:

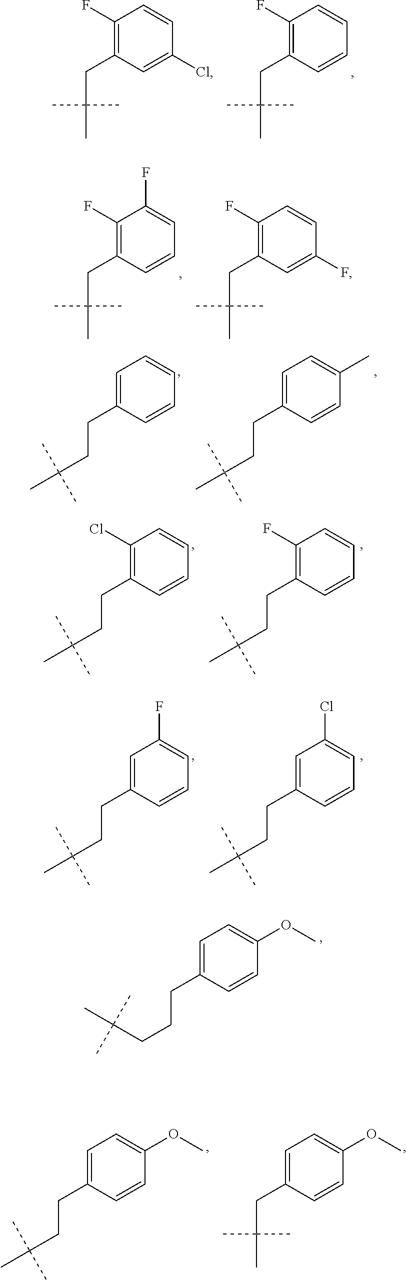

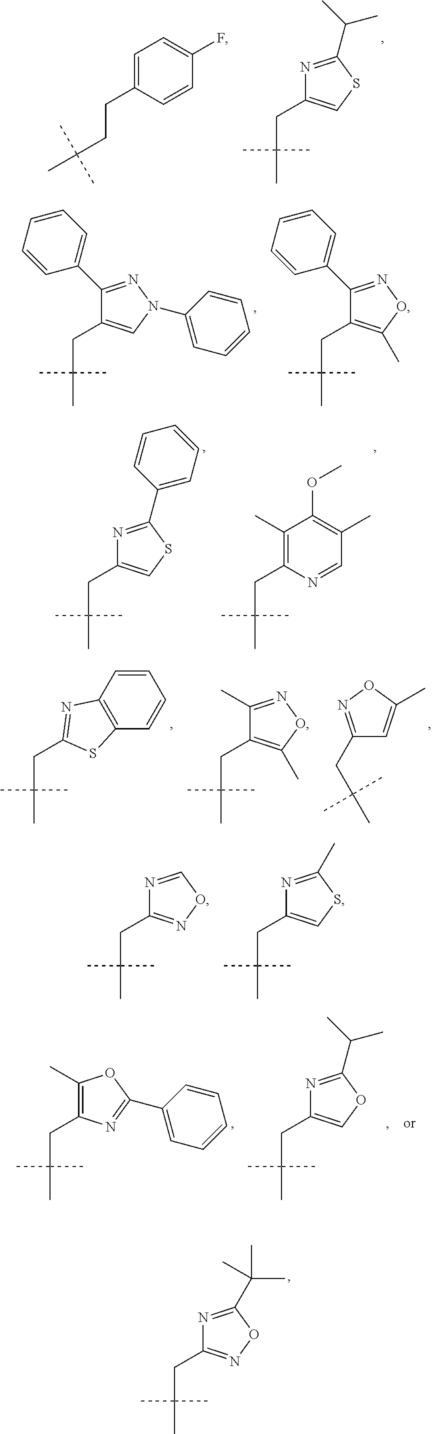

20. The compound according to claim 16, wherein the compound is of Formulae (Ia) or (Ib) 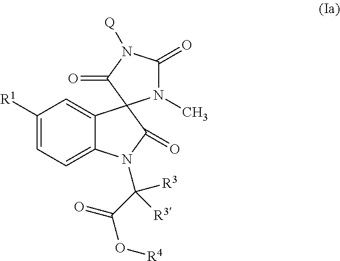
wherein R¹, Q, R³, R³′ and R⁴ are as defined in claim 16; 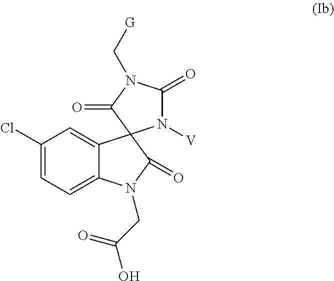
wherein G is Ar or Het,
and wherein V is an alkyl having 1 to 6 carbon atoms;
or derivatives, enantiomers, diastereoisomers, tautomers, salts, solvates, and mixtures thereof.

21. The compound according to claim 16, wherein said compound is selected from the following group:

| Example | Formula |
|---|---|
| 2a | 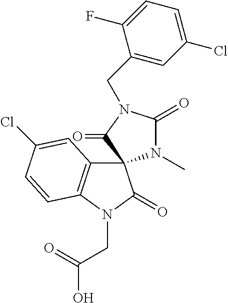 |
| 2b | 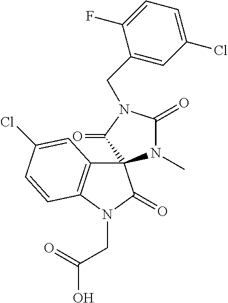 |
| 3a | 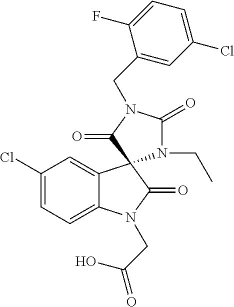 |
| 3b | 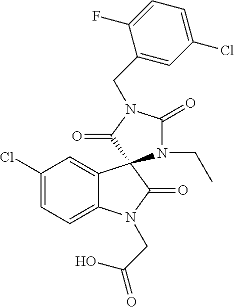 |
| 4a | 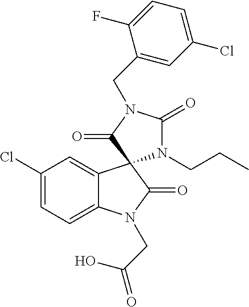 |
| 4b | 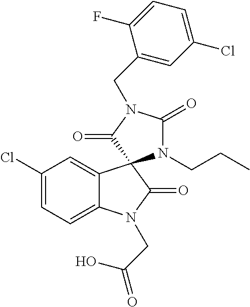 |
| 5a | 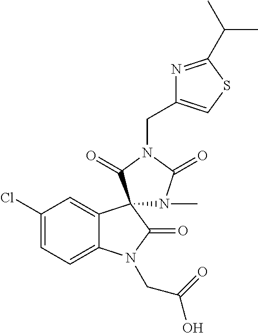 |
| 5b | 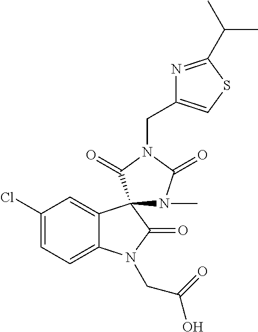 |
| 6a | 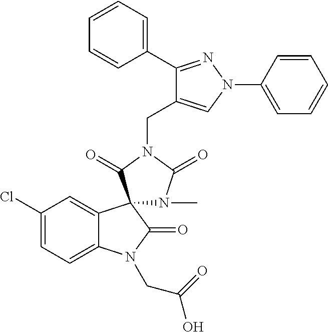 |
| 6b | 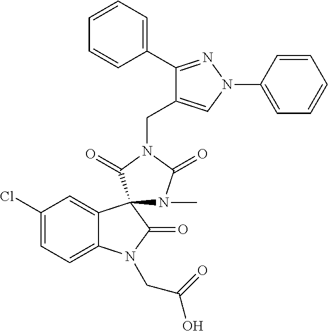 |
| 7a | 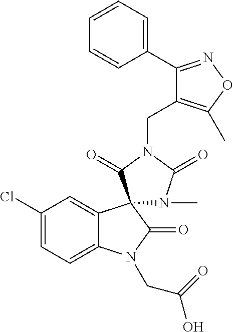 |
| 7b | 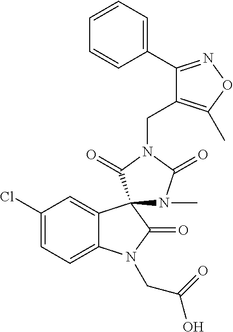 |
| 8a | 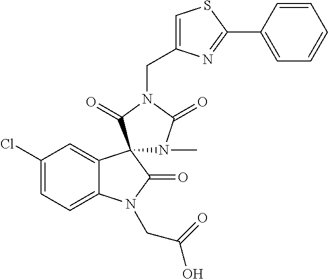 |
| 8b | 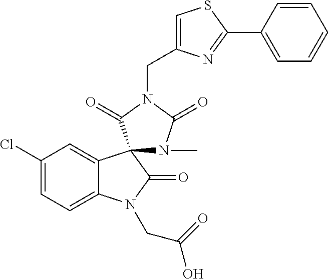 |
| 9a | 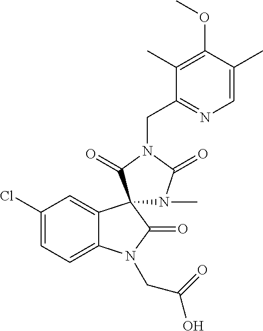 |
| 9b | 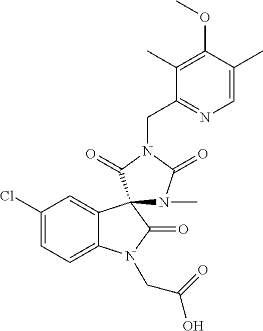 |
| 10a | 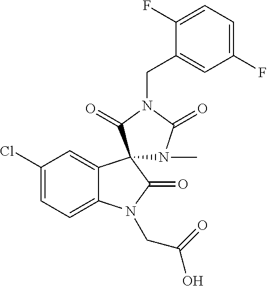 |
| 10b | 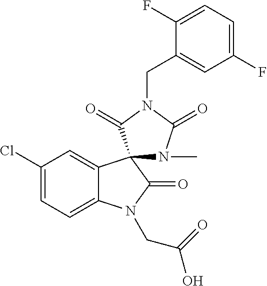 |
| 11a | 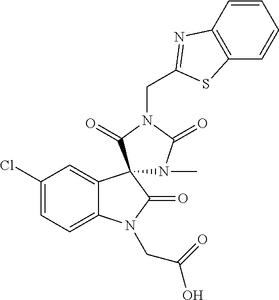 |
| 11b | 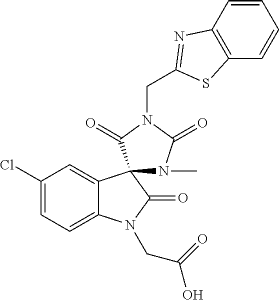 |
| 12a | 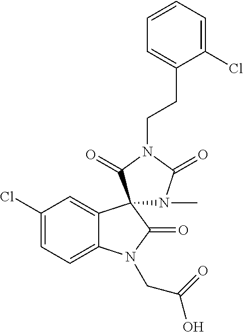 |
| 12b | 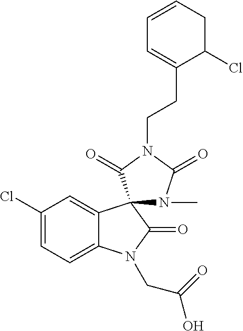 |
| 13a | 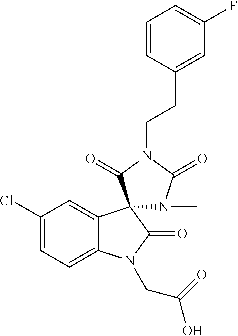 |
| 13b | 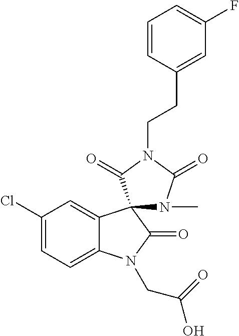 |
| 14a | 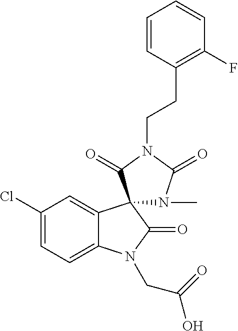 |
| 14b | 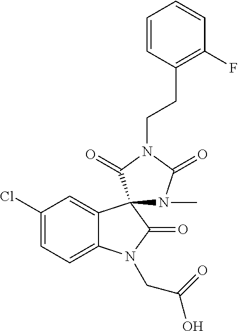 |
| 15a | 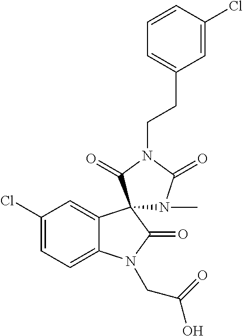 |
| or 15b | 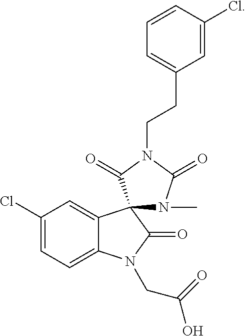 |

22. A method of treating a CRTH2 related disease comprising the administration of a compound according to claim 16 to an individual having a CRTH2 related disease in an amount effective to treat said disease.

23. A method of treating an allergic disease of an inflammatory dermatoses comprising the administration of a compound according to claim 16 to an individual in an amount effective to treat said allergic disease or inflammatory dermatoses.

24. The method according to claim 23, wherein said allergic disease is selected from allergic asthma, allergic rhinitis or allergic conjunctivitis.

25. The method according to claim 23, wherein the allergic disease or inflammatory dermatoses is selected from atopic dermatitis, contact hypersensitivity, allergic contact dermatitis, chronic urticaria/chronic idiopathic/autoimmune urticaria, drug-induced exanthems, photodermatosis or polymorphous light eruption, myositis neurodegenerative disorders, rheumatoid arthritis, multiple sclerosis, osteoarthritis, or inflammatory bowel disease (IBD).

26. A pharmaceutical composition comprising at least one compound according to claim 16 and an excipient and/or adjuvant.

27. The pharmaceutical composition according to claim 26, said composition further comprising at least one additional active ingredient.

28. A kit or a set consisting of separate packs of
(a) an effective amount of a compound according to claim 16 and
(b) an effective amount of an additional active ingredient.

29. A process for producing a compound according to claim 16 comprising the step of reacting a compound of formula (III) with R²—W, 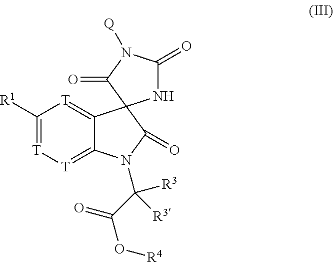
to afford compound of formula (I′) 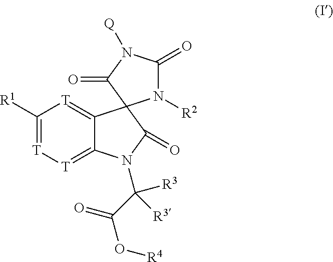
wherein R¹, T, Q, R², R³, R³′ and R⁴ are as defined in claim 16 and
wherein W is a leaving group selected from Cl, Br, I, OMs, or OTf.
